(19) United States
(12) Patent Application Publication
(10) Pub. No.: US 2023/0407379 A1
(43) Pub. Date: Dec. 21, 2023

(54) ELECTRIC FIELD AIDED SAMPLE PREPARATION

(71) Applicant: Agilent Technologies, Inc., Santa Clara, CA (US)

(72) Inventors: Adrienne McCampbell, San Jose, CA (US); Victor Lim, Pacifica, CA (US); Sean Nasri, Los Altos, CA (US); Brian Phillip Smart, San Jose, CA (US)

(21) Appl. No.: 18/246,651

(22) PCT Filed: Jul. 30, 2021
(86) PCT No.: PCT/US2021/044011

§ 371 Date: Mar. 24, 2023

Related U.S. Application Data
(60) Provisional application No. 63/085,319, filed on Sep. 30, 2020.

(51) Int. Cl. C12Q 1/6841; C12Q 1/6806

(52) U.S. Cl. CPC C12Q 1/6841; C12Q 1/6806

(57) ABSTRACT

The present methods and apparatus apply an electric field to a sample in order to prepare it for analysis. In some embodiments, an electric field is applied for drying, staining and coverslipping of tissue sections, providing improved tissue sections for analysis and storage.

### CROSS-REFERENCE TO RELATED APPLICATIONS

This application claims the benefit of U.S. Provisional Patent Application No. 63/085,319, filed Sep. 30, 2020, the contents of which are incorporated herein by reference in their entirety.

### TECHNICAL FIELD

The present invention generally relates to preparation of samples, such as tissue sections for analysis, including drying, staining and coverslipping of biological samples, including but not limited to human and animal tissues as well as cultured cells, cytology specimens, cell smears and generally speaking any cell preparation.

### BACKGROUND

In histology, pathology and other fields, a biological sample such as cellular tissue is collected from a human or animal and then subjected to various processing steps in preparation for or as part of various analytical procedures. Some of the steps typical in processing a biological sample include fixing, embedding, sectioning, drying, staining or conducting other assays, mounting, and cover slipping.

### SUMMARY

According to one embodiment, a method for preparing a sample includes applying an electrical field to the sample effective to accomplish one or more of the objectives disclosed herein.

According to another embodiment, a tissue preparation device includes an electric field-generating device configured for applying an electrical field according to any of the methods disclosed herein and/or to accomplish one or more of the objectives disclosed herein.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a schematic perspective view of a tissue sample preparation device (or paraffin removal device) according to an embodiment.

FIG. 2 is a schematic elevation view of the tissue sample preparation device illustrated in FIG. 1, illustrating its operation.

FIG. 3 is a schematic view of a tissue sample preparation device according to another embodiment.

FIG. 4 is a schematic view of a tissue sample preparation device according to another embodiment.

FIG. 5 shows an embodiment of the present methods in which water trapped between a tissue section and a slide is removed.

FIG. 6 shows samples that were prepared for immunohistochemistry analysis by applying an electric field for antigen recovery.

FIGS. 7 and 9 show samples that were prepared for immunohistochemistry analysis by applying an electric field with ionic salts for antigen recovery.

FIGS. 9 and 10 show samples that were prepared for in situ hybridization analysis by applying an electric field.

FIG. 11 shows the FISH HER2/CEN17 results for two patient samples of breast adenocarcinoma treated with pepsin (panels A and C) or an electric field (panels B and D).

FIG. 12 shows stained tissue stabilizing with a flowable cover medium in accordance with an embodiment of the present methods.

FIG. 13 illustrates an embodiment of the present methods in which an electric field is applied to spread a mounting medium over a sample.

FIG. 14 shows a coverslipped sample slide in which an electric field was applied to spread a mounting medium over a sample.

### DETAILED DESCRIPTION

In histology, pathology and other fields, a biological sample such as cellular tissue is collected from a human or animal and then subjected to various processing steps in preparation for or as part of various analytical procedures. Samples are often stored for a potentially long period of time and subsequently examined by an analytical instrument such as a light microscope, electron microscope or imaging or scanning system. As an example, a biopsy or surgical procedure is performed to collect tissue for subsequent study. The collected tissue is then placed in a container containing a chemical fixative such as formalin to prevent or reduce the natural degradation process. Fixatives crosslink proteins and destroy the functionality of enzymes that degrade tissue. The container can be routed for further processing. Often, a tissue sample is routed for grossing. A technician (for example, a pathologist or other appropriately trained person) examines fixed tissue and selects appropriate portion(s) of the tissue for further examination. The tissue portion(s) are cut to a size that fits easily within the tissue cassette(s). A typical tissue cassette has a volume of about 7.8 cm³, a hinged lid, and flow-through slots or holes to allow the tissue to be immersed in liquid while remaining securely contained inside the tissue cassette. The tissue cassette may then be immersed in a fixative bath for several hours.

The tissue sample can then be subjected to processing, which may be manual or automated using a suitable processing apparatus. One goal is to completely dehydrate the tissue so that the tissue can be infiltrated with paraffin (or other embedding medium) to make it stiff enough to cut later. The tissue is typically immersed in alcohol baths of successively increasing alcohol concentrations, for example a 70% ethanol bath for fifteen minutes, followed by a 90% ethanol bath for fifteen minutes, followed by a series of 100% ethanol baths for longer times. Some processors include microwave or acoustic methods to speed up the exchange of solvents. Next, the dehydrated tissue is immersed in a xylene (or other clearing agent) bath for twenty minutes to an hour to completely remove the alcohol, as the alcohol is immiscible with paraffin. Then the tissue is infiltrated with melted paraffin (usually at about 60° C.) and then cool the tissue to room temperature.

The technician then collects the closed tissue cassettes containing the tissues and brings them to an embedding station. The embedding station includes a hot melt gun containing melted paraffin and a chill plate. The technician opens a tissue cassette and selects a mold that will comfortably fit the tissue inside the tissue cassette. The technician places a small amount of paraffin in the base of the mold, and then arranges the tissue in the mold as the paraffin solidifies on the chill plate. The tissue is oriented so that the tissue closest to the bottom of the mold will be cut first by a microtome. The technician then fills the rest of the mold with melted paraffin. Next, the technician places the backside of the tissue cassette against the paraffin and may add a further amount of paraffin. The tissue cassette can carry a barcode or other information and act as a holder for the tissue block. The technician then sets the mold aside until the paraffin hardens, and then removes the tissue block from the mold. The resulting tissue is referred to as formalin fixed paraffin embedded (FFPE) tissue.

A technician can then use a microtome to cut or slice the tissue block to obtain one or more thin slices of the stiffened tissue, which are referred to as tissue sections. Usually the thickness of these tissue sections is on the order of 4 to 6 micrometers (μm), although a range of 1 micrometer to 30 micrometers is not uncommon. After trimming away the excess paraffin on the top of the tissue block, the technician cuts several sections, which tend to form a ribbon. The ribbon is carefully placed in a heated water bath to flatten both the paraffin and the tissue. The technician then singulates the ribbon into individual sections and draws up each section onto a glass microscope slide. At this point, each slide has one or two or several sections of tissue and paraffin (both the infiltrating paraffin and the embedding paraffin) held onto a major surface of the slide by surface tension from a very thin film of water. Each slide can be barcoded or otherwise marked for identification.

For most staining protocols, tissue sections need to be carefully dried on the slide, as it is important that the sections fully adhere to the slides. However, removing the section from the water can leave a variable layer of water trapped between the section and the glass slide. If the slide is not fully dry before it is baked, the section may not adhere to the glass or may contain wrinkles. Both events are likely to interfere with a successful staining process. A typical method used to remove trapped water is to air dry the slides for about 20-60 minutes in a vertical orientation to allow water to flow to the bottom of the section and evaporate. Other water removal methods include actively passing air over the slide to aid in evaporation of water or tapping the slides to force the trapped water out. These methods can help to decrease the time needed for the water to be removed but still require an extended time to ensure dryness.

After drying, the slide having a tissue section is baked at about 60° C. Usually the slide is placed vertically on a hot plate or in a heated chamber (for example, an oven) for about 20-60 minutes. There are some variants to the heating apparatus available, but all of them essentially involve the use of heated chambers or hot plates in some form. Drying and baking are done to ensure adhesion of the tissue to the slide throughout the staining process and throughout subsequent storage, which may be desired for a decade or longer. Tissue that separates from the slide is lost, the consequence of which can be serious such as in the case of a patient who experienced surgery to obtain the tissue sample. Tissue may not adhere well to non-charged slides, however it is routinely performed for H&E stained sections. To increase adhesion, some laboratories have used positively charged slides so that the negative charges on the proteins and nucleic acids (deoxyribonucleic acid or DNA, and ribonucleic acid or RNA) of the sample interact with the positively charged slides. Other laboratories put an adhesive in the water bath used for mounting samples on plain glass slides in an attempt to ensure that the tissues adhere to the slides. The length of drying and baking time can vary, depending on the subsequent staining process to be performed. The baking protocol is longer for slides that will be stained for immunohistochemistry (IHC) than for the standard hematoxylin and eosin (H&E) staining because IHC is a more aggressive chemical procedure and hence increases the likelihood of tissue sections falling off the slides.

After the tissue has been adhered to the slide, the tissue may be stained. There are many kinds of staining that may be performed on a tissue section. For example, a tissue section may be mounted on a slide for H&E staining. Hematoxylin stains nucleic acids blue and thus is useful as a marker of the cellular nucleus. Eosin stains proteins pink and thus is useful as a marker of cellular membranes, cytoplasm and extracellular matrix. Pathologists often use H&E stained slides to look at the morphology of the tissue structure. In some instances, the pathologist can obtain a diagnosis from studying H&E stained slides without further analysis.

Before staining a tissue section, the paraffin adhered to the slide and intermixed with the tissue is removed. The traditional sequence of steps involved in removing the paraffin is essentially the opposite of that described above for applying paraffin to the sample. The slide is dipped in xylene or another clearing agent to dissolve the paraffin and remove it. Other solvents such as isopropyl alcohol can be used for paraffin removal, though they generally do not work as well as xylene. The slide is then placed into a series of ethanol solutions having decreasing concentrations, starting with 100% ethanol to remove the xylene, and continuing down to a 70% ethanol/30% water composition to rehydrate the tissue. Then the slide is placed in deionized water.

After removing the paraffin, the slide is stained. For example, the slide may be placed into a hematoxylin solution to stain the nuclei and then rinsed. Subsequently, the slide may be placed into an eosin solution (to stain protein in the sample) and then rinsed. Next, a mounting solution is placed over the stained tissue and a thin coverslip (usually very thin glass or plastic) is placed over the tissue and the edges are bonded to the slide. The coverslip allows for easier viewing under the microscope.

Some diagnoses or analyses require the use of other types of staining. For example, special stains are used to diagnose microbial infections. As another example, immunohistochemistry (IHC) refers to using antibody-based reagents to test for the presence of specific antigens on various polypeptides. It is often used to characterize cancers with greater specificity. IHC staining processes are generally similar to H&E, in that the paraffin needs to be removed and the tissue rehydrated.

However, there is usually an extra step for IHC in which the antigen in the tissue (particularly the antigen to be bound by the antibody reagent) is “retrieved” by heating the tissue to a desired temperature in a suitable buffer. Once the antigen is retrieved, the antibody is applied. The slide is then washed, and a labeling step is performed to apply a color stain to the slide where the antibody has stuck to the tissue. Loosely defined, heat-induced epitope retrieval (HIER) is a procedure often used prior to immunohistochemistry staining to improve staining by modifying the molecular conformation of polypeptides containing target antigens, generally returning them to their original or “pre-fixative” conformation. The HIER procedure includes an exposure of slide-mounted specimen material (sectioned tissue and other cellular preparations) to a heated buffer solution. Alternative terms such as “protein unmasking”, “decloaking”, “antigen retrieval” and “epitope recovery” have also been used to describe these procedures. This retrieval process is used because, although aldehyde-based fixatives are excellent for preserving cellular morphology, they can also cause polypeptide cross-linking, resulting in the inability of some antigens to bind complementary antibodies. Heat causes cross-linked antigens to unfold (in manner similar to DNA denaturation), while buffer solutions aid in maintaining the conformation of the unfolded protein. The primary differences between various HIER methods are the means by which such solutions are heated and exposed or applied to slides. HIER is commonly used in conjunction with enzyme digestion as a means of improving the reactivity of various antigens within IHC staining reactions.

Household-grade microwaves are often used for HIER but have several shortcomings, such as difficulty in regulating temperature, the likelihood of boiling, and the fact that these units loose power over time. Vegetable steamers, like water baths, are often incapable of heating solutions above 95° C., resulting in exceptionally long HIER protocols. In addition, vegetable steamers do not hold very many slides, relative to other devices. Pressure cookers have the advantage of higher operating temperature and a closed system design, which allows for shorter protocols and eliminates the potential for boiling. Units that are retrofitted with a digital control panel and pressure gauge provide for better temperature regulation and permit the operator to monitor performance during the procedure. Although household appliances are commonly used within the laboratory environment, scientists agree that more standardization and specially-designed instruments are desired.

There are several commercially available devices designed specifically for HIER. For example, Pick Cell Laboratories' has introduced the 2100 Retriever™, which is a pressure cooker-like device (without the pressure gauge and time/temperature display found on similar units). The EZ Retriever™, from BioGenex Laboratories, is essentially an industrial-grade microwave oven containing four Teflon® reaction chambers and a temperature-monitoring probe. Like the devices upon which their operations are based (i.e. pressure cookers and microwave ovens), these new instruments possess many of the same shortcomings. The PT (Pre Treatment) Module™ from Lab Vision Corporation is a semi-automated instrument designed primarily for use with Lab Vision's Autostainer™ slide racks, which allows slides to be transferred directly from the PT Module™ to an Autostainer™ at the completion of the HIER procedure. The primary shortcomings of this device are that it requires the operator to fill and drain reagents from two large, removable stainless steel ‘tanks’, and consumes a disproportionately large volume of retrieval solution.

There are a variety of IHC stainers on the market, some of which offer HIER capability. Unlike the devices described above, in which many slides are submerged in a heated buffer solution at the same time, HIER performed within IHC stainers usually involves processing slides individually. For example, Ventana Medical Systems' Benchmark® stainer essentially ‘sprays’ HIER solution unto individual, horizontally-oriented slides within a rotary ‘carousel’, then heats each slide to a preset temperature, and rotates the carousel in order to repeat this process on other slides. In most protocols, the HIER solution is applied as many as nine times before HIER is considered completed. In a similar fashion, Vision-Biosystems' Bond-maX™ stainer dispenses HIER reagents onto rows of horizontally-arranged slides via a pipettor mechanism, and then whole rows are heated together.

The addition of HIER capability to an IHC slide stainer increases the likelihood of specimen loss and drives up the cost of performing this otherwise ‘inexpensive’ procedure. The former problem is especially significant because the operator is usually not aware of specimen loss until an entire IHC procedure has been completed, which is expensive, and then the operator is required to repeat the IHC stain.

Another important aspect of HIER is the cost-effectiveness of various methods and devices. This is especially true when one considers that addition of HIER, or deparaffinization and HIER, to an automated protocol can substantially increase the overall cost of IHC. An important characteristic of HIER devices is the amount of reagent that is consumed during the HIER procedure, and there are significant differences between devices. For example, some studies have demonstrated that the cost of performing HIER on an automated IHC stainer can be as much as six times greater than performing HIER in a modified pressure cooker.

In their simplest form, most HIER devices consist of a primary chamber into which secondary reagent containers are placed, surrounded by a reliable mechanism for heating the liquid in the secondary containers. The limit to the number of slides (secondary reagent containers) that such a device can hold is the size of the primary chamber. Considering the importance of producing consistent results, a desirable HIER device: A) incorporates a precision-controlled heat source, capable of maintaining temperatures at or above 100° C.; B) holds a reasonable volume of retrieval buffer and slides; and C) minimizes the potential for evaporation and boiling of the HIER solution. These latter requirements are important because excessive evaporation: A) causes the salt concentration of the buffer to fluctuate; B) boiling can cause specimen material to be released from the slide; and C) boil-over can lead to exposure of ‘raw’ specimen material to the atmosphere, resulting in inadequate retrieval and less than ideal morphology, due to drying artifact. As a function of the inverse relationship between temperature and exposure time, devices that operate above 100° C. and prevent boiling (for example pressure cookers) have become very popular because they produce good results in the shortest possible timeframe.

Another type of staining is in situ hybridization (ISH), which uses labeled complementary DNA or RNA probes to localize a specific DNA or RNA sequence in a sample. In situ hybridization assays can be used to quantitatively determine the presence or absence of gene amplification in frozen tissues or formalin-fixed, paraffin embedded tissue specimens (whole tissue, cell pellets or cell smears) using fluorescent, silver or chromogenic labeled nucleic acid probes. This staining can be used to determine genetic markers in cancer that are relevant to the selection of therapeutics for treatment of the disease. Following ISH, the slide is covered using a DAPI mounting media, which is a fluorescent blue stain for adenine-thymine rich regions in DNA.

Before applying the labeled nucleic acid probes, tissue samples are usually subjected to proteolytic digestion. For frozen tissue, the tissue may be cut using a cryostat, followed by fixation of tissue and then proteolytic digestion prior to labeled probe hybridization. For FFPE tissue, paraffin is removed from a tissue section that has been cut and adhered to a glass slide (as described above). After deparaffinization and rehydration, samples undergo a heated pretreatment step in aqueous buffer which eases subsequent protease digestion by breaking the formalin-induced disulphide bonds. Then a proteolytic digestion is performed using proteases such as pepsin or proteinase K to digest proteins or break peptide binding to ease access of the labeled nucleic acid probes to the genomic target DNA. The proteolytic digestion also reduces autofluorescence generated by intact proteins. The enzymatic digestion procedure may be adapted to the tissue fixation time and varies by temperature and time, for example from 30 seconds to up to 50 minutes. The proteolytic digestion enhances permeabilization of the cytoplasm to allow nucleic acid (RNA or DNA) probes access to tissue nucleic acids. In properly digested cells, the autofluorescence is mainly restricted to the nuclei and the level does not disturb red and green probe signals.

Current ISH methods using proteolytic digestion have several disadvantages. Proteolytic digestion tends to be the most variable step in ISH due to variations in preanalytic fixation methods between different laboratories and tissue types. Under-digestion and over-digestion can occur very frequently and require a repeat of the ISH assay. In the absence of proteolytic digestion ISH, results are sub-optimal with high levels of autofluorescence, irregular DAPI nuclear staining patterns and poor signal distribution of the probes. Heavily under-digested samples are often characterized by green autofluorescence in the cytosol and extracellular matrix. Probe hybridization is hampered by the high presence of proteins and peptide chains, which reduces signal intensity because of higher autofluorescence and lower probe hybridization, thereby reducing the signal-to-noise ratio. The reference probe signal may result in the presence of green signals despite sub-optimal under-digestion of the sample. Over-digestion results in insufficient DNA DAPI complex formation and makes the nuclei staining appear heterogeneous. This staining pattern is referred to as doughnut formation or damaged nuclear membranes. Intense over-digestion results in empty “ghost” nuclei and destroyed nuclear morphology. These artifacts can require repeat testing as well as affect pathologist interpretation of the score and therefore affect patient treatment.

Multiplex IHC staining refers to immunohistochemical (IHC) staining of a sample with antibodies for multiple antigen targets on a single slide. Multiplex staining has multiple IHC benefits that include increased value per slide, simplified procedures and reduced turnaround time. Multiplex multi-polymer (or chromogen) detection simplifies protocols by reducing the number of steps. Applications for multiplex IHC include (1) clinical assessment of a panel of protein markers deemed to be relevant for disease diagnosis or treatment, and (2) clinical assessment of tumor microenvironment constitution or detection of immune cells infiltrating to tumor cells, which informs choice of immunotherapy or standard of care treatment for many cancers. In some exemplary instances of application (2), identification of immune cells or macrophages that appear to be very similar in morphology to tumor cells is very desirable. The distinction between B and T cells amongst tumor cells is important for immunotherapy choice. Immune cells and tumor cells can be distinguished using multiplex IHC comprising antibodies specific to proteins expressed only by immune cells or only by tumor cells.

A typical workflow for multiplex IHC on a tissue section can include some or all of the following steps.
1. Deparaffinize slides.
2. Hydrate tissue section in a series of graded alcohols to water.
3. Block endogenous peroxidases that could increase background staining.
4. Antigen Retrieval by heating the tissue section to 95° in a high pH buffer containing a salt.
5. Protein Block to prevent non-specific proteins from binding to IHC antibodies.
6. Apply Primary Antibody #1 which binds to its specific antigen (if any) in the tissue specimen.
7. Bind HRP secondary antibody around primary antibody #1: horseradish peroxidase (HRP)-coupled polymer bind to the primary antibody #1.
8. Polymer/Chromogen: precipitate chromogen (DAB, HRP Magenta or other chromogens) around primary antibody #1.
9. Denature available antibodies bound to tissue: Addition of a denaturing reagent such as sulfuric acid will denature primary antibody #1 without affecting retrieved antigens that are still available.
10. Counterstain with hematoxylin for identification of nuclei. This step is optional at this point and can be performed following binding of primary antibody #2 to its antigen and conjugation to secondary antibody and chromogen.
11. At this step, the tissue section can be dehydrated in a series of graded alcohols and coverslipped for analysis, such as analysis by scanning by a whole slide imager.
12. Remove coverslips, wash with xylene and rehydrate in a series of graded alcohols to water.
13. Add Primary Antibody #2 which binds to its specific antigen (if any) in the tissue section.
14. Bind HRP secondary antibody around primary antibody #2: horse radish peroxidase (HRP)-coupled polymer binds to the primary antibody #2.
15. Polymer/Chromogen: precipitate chromogen (DAB, HRP Magenta or other chromogens) around primary antibody #2.
16. Counterstain with hematoxylin for identification of nuclei.
17. Dehydrate the tissue section and mount a coverslip on the tissue section using mounting medium.

From the foregoing, it is evident that the processing of collected tissue for subsequent study involves many steps and a considerable amount of time. Thus, any improvements in such processing that eliminate one or more of these steps and/or reduce the amount of time required would be desirable.

To address the foregoing problems, in whole or in part, and/or other problems that may have been observed by persons skilled in the art, the present disclosure provides methods, processes, systems, apparatus, instruments, and/or devices, as described by way of example in implementations set forth below. It is contemplated that any of the various embodiments of the present methods, processes, systems, apparatus, instruments, and/or devices can be combined with one or more of the other embodiments.

As used herein, the term “solid substrate” refers to any sample holder, support or substrate having at least one surface for a biological or chemical sample. Solid substrates include, but are not limited to, a slide, a plate, a frit, beads, a porous medium, a filter, and a container. Thus, the solid substrate can be a carrier, test tube, chip, array, or disk that can support at least one sample. In some embodiments, the solid substrate is a slide. A slide typically has first and second major slide surfaces, which may be flat or curved. Slides and other solid substrates can be made of glass, such as soda lime glass, borosilicate glass or fused quartz, or transparent plastic, or other transparent or semi-transparent materials.

As used herein, the term “sample” means a biological, chemical, industrial or sample for which analysis is desired, such as a tissue sample, blood sample, or cellular sample. In some embodiments, a tissue sample may be a sample of a tissue biopsy obtained from a patient. Biopsies of interest include both tumor and non-neoplastic biopsies of skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc.

As used herein, the term “embedding medium” refers to paraffin and similar materials which are suitable for embedding a biological sample such as a tissue sample.

As used herein, the term “electrode” refers to any solid structure that carries an electric current such that an electric field arise. Electrodes can comprise any suitable material, such aluminum or other metals. Electrodes can have any desired size or shape, such as an elongated rod-type geometry or a thin planar (plate-shaped) geometry. For example, a suitable electrode can be a metallic (e.g., aluminum) foil. Exemplary electrode include flat electrodes such as a plate and pin electrodes; pairs of electrodes where one or both is stationary or one or both moveable; and electrodes which are configured for operation at room temperature, or at an elevated or reduced temperature.

A non-limiting example of an embodiment of an apparatus for applying an electric field to a sample will now be described.

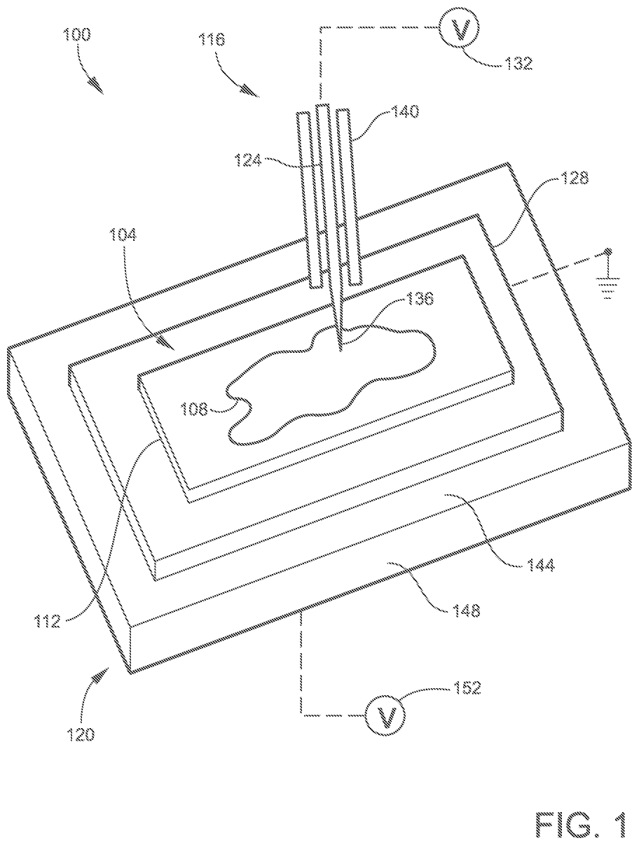

FIG. 1 is a schematic perspective view of an exemplary tissue preparation device 100. The device 100 may also be referred to as an electric field applying device. FIG. 1 also illustrates a supported tissue arrangement 104 that includes a tissue 108, which is exemplary of samples in general and may also be referred to as a tissue sample, disposed on an upper surface of a solid substrate 112 (for example, a microscope slide). The tissue 108 may freely rest on the solid substrate 112 in an unbound manner. For example, the tissue sample 108 may have been collected and processed as described above. The device 100 is configured for applying an electric field to a tissue 108, which may be done in preparation for staining and/or for other desired processing or analysis.

The electric field is important to create the desired effect. Many other means and embodiments can be envisioned for creating the requisite electric field, and would be known to those skilled in the art. That encompasses different dimensions and voltages within the following described exemplary embodiments, as well as other physical means of creating said electric fields (e.g. optical generation, inductive generation through a changing magnetic field, electret-based, etc). Any of these means can be used to generate the required electric fields and remain within the scope of the invention herein described. Additionally, the invention is not limited to the exemplary field strengths herein described, but larger or smaller fields can be envisioned by one skilled in the art for different material and platform choices.

The electric field applying device 100 includes an electric field-generating device 116, and may further include a heating device 120. The electric field-generating device 116 includes an electrode arrangement, such as one or more movable or stationary first electrodes (or discharge electrodes) 124 and one or more (typically stationary) second electrodes (or counter-electrodes) 128, and an appropriate voltage source (or electrical power supply) 132. The voltage source 132 may be a direct current (DC) voltage source or alternating current (AC) voltage source. In some embodiments, the voltage source 132 is an AC voltage source, which may be a high-frequency voltage source such as a radio-frequency (RF) voltage source or a microwave-frequency voltage source. At least the first electrode 124 is in electrical communication with the voltage source 132. Depending on the embodiment, the second electrode 128 may be in electrical communication with the voltage source 132 or may be coupled to electrical ground. In some embodiments, the voltage source 132 is a high-voltage source capable of applying a DC voltage potential to the first electrode 124 on the order of kilovolts (kV). For example, in some embodiments, the voltage potential (relative to ground) may be in a range (in absolute value) from about 4000 V (4 kV) to about 30,000 V (30 kV), including all of the voltages within this range with non-limiting, example electric fields in the range of 1.5 MV/m to 8 MV/m. The voltage potential applied may be positive or negative, i.e., the range may be from about +4000 V to about +30,000 V or from about −4000 V to about −30,000 V. As a non-limiting example, the spacing of the electrodes can be set to generate an electric field in the range between −1.5 MV/m to −8 MV/m or +1.5 MV/m to +8 MV/m. The foregoing range is merely one example. In some embodiments, the voltage potential may be greater than 30,000 V yielding an electric field in excess of 30 MV/m. In some embodiments, where a corona discharge is not necessary, the voltage potential may be less than 1000V yielding electric field less than 3 MV/m. More generally, the voltage source 132 is capable of applying a DC or AC voltage to the first electrode 124 at a magnitude (or in the case of AC power, a peak-to-peak amplitude and frequency) that is sufficient or effective to strike and maintain a corona discharge or plasma in the environment in which the first electrode 124 and the tissue 108 are located.

In some embodiments, the first electrode 124 may include a (highly) curved feature configured to generate a region of elevated electric field strength surrounding the first electrode 124. For example, the curved feature may be a sharp or geometrically abrupt feature, such as an edge or a pointed tip, or a small-diameter wire. In the illustrated embodiment, the first electrode 124 is configured as an elongated rod that terminates at a distal electrode tip 136. The first electrode 124, or at least a tip section thereof that terminates at the distal electrode tip 136, may be tapered such that the distal electrode tip 136 is sharp or pointed. Generally, a sharper distal electrode tip 136 generates a stronger electric field at the distal electrode tip 136, as compared to a more blunt geometry. Thus, in some embodiments, the first electrode 124 may be configured as a needle or pin (for example, a corona discharge needle). In some embodiments, the first electrode 124 may be coaxially surrounded by a body 140 (shown in cross-section in FIG. 1) composed of an electrically insulating material. The electrically insulating material may also be a sufficiently thermally insulating material, or the body 140 may further include a thermally insulating material coaxially surrounding the electrically insulating material. The insulating body 140 may be configured to be hand-held by a user. That is, the first electrode 124 may be configured as a handpiece that is held by a user like a pen. Alternatively, the insulating body 140 may be configured to be mounted to an automated device (for example, a motorized stage or robot). Thus, depending on the embodiment the first electrode 124 may be movable relative to the tissue 108 in a manual or automated manner.

The second electrode 128 is typically configured to serve as a counter-electrode or ground plane. The second electrode 128 may be positioned so as to cooperate with the first electrode 124 in defining the location and spatial orientation of the electric field and plasma generated by the applied voltage. In some embodiments and as illustrated, the second electrode 128 is positioned on a side of the tissue 108 opposite to the first electrode 124. In other words, the tissue 108 is positioned between the first electrode 124 and the second electrode 128. In other embodiments, the second electrode 128 may be positioned above the tissue 108, or juxtaposed at roughly the same elevation as the tissue 108 relative to some reference plane. In the illustrated embodiment, the second electrode 128 has a thin planar (plate-shaped) geometry of greater planar area than that of the supported tissue arrangement 104, and the supported tissue arrangement 104 is placed onto the second electrode 128. For example, the second electrode 128 may be a metallic (for example, aluminum) foil. In some embodiments, the second electrode 128 may have an elongated rod-type geometry similar to the illustrated example of the first electrode 124. In some embodiments, more than one first electrode 124 and/or more than one second electrode 128 may be provided.

In some embodiments, particularly when AC power is utilized, a second electrode may coaxially surround a first electrode such that an electrically insulating portion of the body is interposed between the first electrode and the second electrode. In this case, the second electrode may in turn be coaxially surrounded by electrically insulating and/or thermally insulating material.

The device 100 may include electronics that include the voltage source 132 and other appropriate components. The electronics may include, for example, an ON/OFF switch (not specifically shown) for controlling the application of voltage potential to the first electrode 116, a component (for example, a control knob, not shown) configured for adjusting the level of voltage potential applied to the first electrode 116, etc. Some or all of the electronics may be arranged in a control console of the device 100. In hand-held embodiments, the ON/OFF switch (or both the ON/OFF switch and voltage level adjustment component) may be located at the insulating body 140 so as to be easily accessible by the user. Alternatively, controls such as the ON/OFF switch and voltage level adjustment component may be located at the control console or a foot-operated module.

The heating device 120 generally may have any configuration effective for generating and transferring heat energy to an upper heating surface 144 of the heating device 120. Thus, for example, the heating device 120 may include a body 148 containing one or more resistive heating elements (not specifically shown) in thermal contact with the heating surface 144, and a voltage source (power supply) 152 providing electrical current to the heating elements. Thus in the illustrated embodiment, the second electrode 128 is placed or mounted on the heating surface 144 of the heating device 120, and the tissue 108 and supporting substrate 112 are in turn placed or mounted on the second electrode 128. In the present embodiment, the device 100, including the heating device 120, has an open architecture. Alternatively, the heating device 120 may include a chamber in which the first electrode 124, the second electrode 128 and the heating surface 144 are positioned, and into which the tissue 108 and supporting substrate 112 are loaded. As an alternative to resistive heating elements, the heating device 120 may provide one or more radiant heating sources such as infrared (IR) lamps.

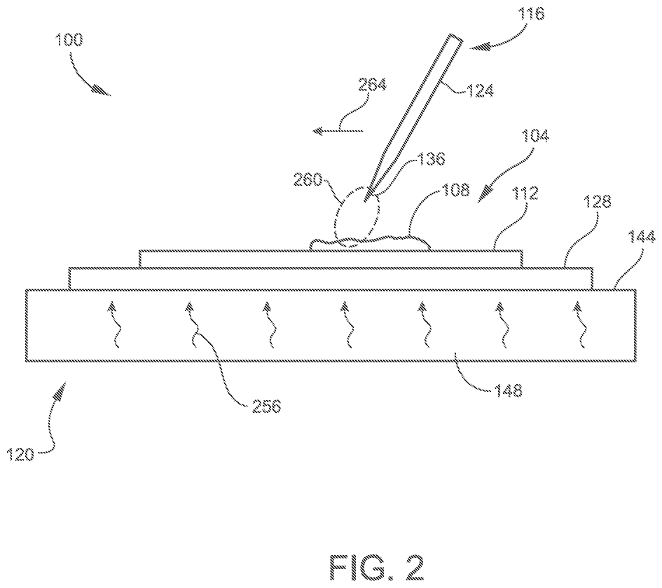

FIG. 2 is a schematic elevation view of the tissue preparation device 100, illustrating its operation. The tissue 108 and supporting substrate 112 are positioned such that they are in thermal contact with the heating surface 144 of the heating device 120, such as by being placed on the second electrode 128. The heating device 120 is then activated to generate and transfer heat energy 256 to the heating surface 144, and consequently to the tissue 108 via heat conduction. A sufficient amount of heat energy 256 is deposited in the tissue 108 to accomplish one or more objectives in preparing the tissue 108 for analysis (as described below).

In some embodiments, the electric field-generating device 116 is then activated to generate an electric field of a strength great enough to generate and sustain a corona discharge or plasma 260 in a region around the electrode tip 136 of the first electrode 124, but not so great as to cause electrical arcing between the first electrode 124 and another object such as the second electrode 128. The electric field accelerates free electrons in the air (or other gaseous medium) into collisions with neutrals (neutral atoms and molecules) in the air (or other gaseous medium). Some of the collisions occur at a high enough energy to ionize the impacted neutrals, thereby liberating more electrons and leading to more collisions between free electrons and neutrals. As long as the electric field is present and of sufficient strength, the ionization events continue in a chain-reaction effect termed an electron avalanche. Photons are also generated in the plasma 260 due to recombination events between electrons and positive ions, and contribute to ionization of neutrals as well. The plasma 260 generated by the electric field-generating device 116 generally is a mixture of charged particles (ions and electrons) and neutrals, as well as other energetic species such as metastables and photons.

In some embodiments, the first electrode 124 has a positive polarity relative to the second electrode 128. In this case, the plasma 260 may be a positive corona discharge. Positive ions are repelled from the first electrode 124 and drawn toward the second electrode 128. On the other hand, negative ions are drawn toward the first electrode 124 and repelled from the second electrode 128. Similarly, electrons are drawn toward the first electrode 124 and repelled from the second electrode 128. In other embodiments, the first electrode 124 may have a negative polarity relative to the second electrode 128, in which case the plasma 260 may be a negative corona discharge.

In FIG. 2, the closed dashed line referred to herein as the plasma 260 schematically depicts the outer spatial extent of the plasma 260 (or at least the active plasma), which may also be referred to as the ionization or plasma-forming region. Outside of this region (plasma 260), the electric field is not strong enough to sustain plasma in the air (or other gaseous medium). In other words, plasma is extinguished outside of this region. It will be understood that the plasma 260 is depicted schematically for illustrative purposes. In practice, the actual size and shape of the plasma 260 (for example, cloud, plume, etc.) may differ appreciably from the schematic depiction shown in FIG. 2.

As depicted in FIG. 2, the energized first electrode 124 is positioned above the tissue 108, and sufficiently close to the tissue 108 that the tissue 108 is exposed to the highly energetic plasma 260. Consequently, the energetic species of the plasma 260 interact with the sample. The first electrode 124 can be moved in any desired direction across (over) the tissue 108 and the underlying substrate 112 to apply an electric field to accomplish one or more of the objectives described herein. For example, in some embodiments, movement of the first electrode 124 in a given direction will either push or pull trapped liquid or a mounting medium in a desired direction. As one non-limiting example, FIG. 2 depicts the first electrode 124 being moved over (without contacting) the tissue 108 in the left-ward direction (from the perspective of FIG. 2), as indicated by a horizontal arrow 264.

A non-limiting example of a method for preparing a tissue will now be described, using the example described above and illustrated in FIGS. 1 and 2. A paraffin-embedded tissue 108 is provided. Providing the tissue 108 may include various processing steps after initially acquiring the tissue 108 from the source, such as fixing, dehydration, alcohol removal, paraffin infiltration/embedding, sectioning, etc. as described herein. In some embodiments, providing the tissue 108 includes positioning (placing or mounting) the tissue 108 on a solid substrate 112, and positioning (placing or mounting) the tissue 108 (supported on the substrate 112) on or near a (second) electrode 128. Heat energy 256 is applied to the tissue 108 as desired, for example, to aid in antigen retrieval. An electric field is applied that is effective to apply an electric field to the tissue 108. In this example, the electric field is generated between a first electrode 124 and the second electrode 128 by applying a voltage potential to the first electrode 124 generating an electric field dependent on the electrode spacing. Further in this example, the electric field applied is effective to generate plasma 260 in a region extending at least between the electrode tip 136 of the first electrode 124 and the tissue 108. The first electrode 124 is moved in one or more directions relative to the tissue 108 as desired, to apply the electric field in a repeated manner. Typically, the first electrode 124 is moved while the tissue 108 remains stationary. Alternatively, or additionally, the tissue 108 may be moved relative to the first electrode 124.

Before, during, and/or after applying an electric field to a sample such as the tissue 108, a staining process or other preparation process may be performed on the tissue 108. For example, the tissue 108 may be stained with a standard staining reagent such as hematoxylin and eosin (H&E), or an immunohistochemical (IHC) staining reagent or other special staining reagent. As another example, the tissue 108 may be treated with a mounting medium and/or fluorescent stain (such as a DAPI mounting medium) after application of an electric field. Other desired processes may be performed on the tissue 108. For example, nucleic acid probes may be applied to the tissue 108 for performing in situ hybridization analysis. As another example, nucleic acids may be isolated from the tissue 108 and subjected to further processing such as, for example, amplification by polymerase chain reaction (PCR), hybridization, etc.

As one aspect, the present technology provides methods and apparatus for drying a sample on a substrate surface and/or other liquid by applying an electric field will now be described. This technology can reduce the time necessary to ensure samples are dry before baking. By way of example, a laboratory which normally leaves slides out to dry for 60 minutes can reduce that time to about 5 minutes.

In some embodiments, the present technology provides methods of placing a sample on a solid substrate. The methods can comprise placing a sample containing a paraffin-embedded tissue in a liquid bath; contacting the paraffin-embedded tissue with a solid substrate (such as a slide) so that the sample is spread on a substrate surface; and removing liquid trapped between the substrate surface and the sample by applying an electric field to the trapped liquid. By way of example, the solid substrate can be a slide having first and second slide surfaces, and the sample can be a tissue section. The methods can also comprise moving the slide so that the slide surface is substantially vertical when the trapped liquid is removed. In this context, “vertical” refers to a position with respect to the force of gravity, for example, where the major slide surfaces are parallel to the force of gravity. In some embodiments, a slide surface is substantially vertical so that the tissue has a top edge and a bottom edge, and the first electrode is located below the bottom edge. The first electrode can be moved from the top edge to the bottom edge while applying the electric field, so that the trapped liquid moves toward the bottom edge. Then the first electrode can be returned to a position at the top edge, and the movement of the first electrode to the bottom edge while applying the electric field can be repeated any desired number of times.

Among other advantages, the present drying methods actively use force and energy to draw out the trapped liquid. When water trapped between a tissue and a slide has reached a very thin layer, it takes a lot of energy to remove that water. Previous methods do not have the ability to supply that energy. Tissue sections also come in various thicknesses, typically between 2 microns to 10 microns. For thicker sections, removing trapped water is more difficult because blowing air across the top surface of the tissue does not work as well. In some embodiments, the present drying methods do not have this issue.

In some embodiments, the present methods comprise applying an electric field to remove water trapped between a tissue and a slide. In some embodiments, a large potential is applied to a metal pin at a distance sufficiently close to the trapped water. A reference ground is placed near the pin to create an electric arc. For example, a metal block can be used as the ground plane. A wet tissue slide with water trapped between the paraffin embedded tissue section and the glass slide can be placed vertically between the pin and the ground plane. The block is held at a temperature below the melting point of paraffin. The pin is located near the bottom edge of the tissue section so that gravity and the electric field draw the water in substantially the same direction. The pin can be placed approximately 4 mm away from the slide. In some embodiments, the power supply is turned up to −20 kV, or another suitable voltage, creating a corona discharge and an electric field which can be in excess of 5 MV/m. Once the power supply is turned on, trapped water between the section and the slide finds a path and moves towards the pin. Several arc paths to ground are created. These arcs generate enough energy to evaporate the water. As water evaporates, more water is pulled towards the ground and pin. Since this path can be between the tissue and slide, the water is actively being pulled from underneath the tissue.

In some embodiments of the present method of removing trapped water, the voltage is at least 5 kV, or 10 kV, or 20 kV; and/or the second electrode is a ground electrode; and/or the first electrode is between about 1 mm and about 7 mm away from the tissue, alternatively between about 3 mm and about 5 mm, which can yield an electric field in range of 350 kV/m to 40 MV/m. In some embodiments, the tissue contacts a first slide surface, and the first electrode is positioned facing a second slide surface. In some embodiments, the tissue contacts a first slide surface, and wherein the second electrode is in contact with a second slide surface, and the second electrode is at a temperature below the melting point of paraffin.

As another aspect, the present technology provides methods of preparing a sample for in situ hybridization comprising applying an electric field to a sample comprising a polynucleotide and a polypeptide; and contacting the sample with one or more labeled nucleic acid probes. The sample can be a paraffin-embedded tissue sample (such as whole tissue, cell pellets, or cell smears), and can comprise one or more cells. The polynucleotide of interest may be within a nucleus of the cells. The present technology allows for the binding of ISH probes to target polynucleotides within the sample without proteolytic digestion. In some embodiments, the present methods avoid proteolytic digestion artifacts including high levels of autofluorescence, nuclear artifacts and low levels of probe signals. Applying an electric field may therefore improve quantitation of probe signal and quantitation of assay results. It may also reduce the time required to perform in situ hybridization assay by, in some embodiments, eliminating a proteolytic digestion step. In some embodiments, an electric field is applied at a strength and for a time sufficient to expose the DNA of interest, avoid the step of digesting these cell structures with a proteolytic enzyme (such as pepsin or proteinase K) before the probe can access and bind to the nucleic acids. In some embodiments, the sample can be made substantially free of autofluorescence from intact proteins during analysis of fluorescently labeled probes hybridized to the polynucleotide. In some embodiments, the methods may also comprise removing substantially all paraffin or other embedding medium from the sample before the contacting with the labeled probes, such as by a combination of applying an electric field to the sample and rinsing the sample with a deparaffinization solvent.

In some embodiments, the present methods comprise exposing the DNA in a sample. In some embodiments, this is done without proteolytic digestion. By applying an electric field for DNA exposure, the proteolytic digestion step is not required, and the present methods decrease high levels of autofluorescence, nuclear artifacts and processing time for the assay. A lack of nuclear artifacts will also make scoring or quantifying of probe signal easier and result in more accurate test results. In some embodiments, the present technology improves probe signal and reduces background for some assays. In some embodiments, DNA exposure can be accomplished by exposing the tissue to an electrical field lesser in magnitude than required to generate a corona discharge. For example and without limitation, 500V at a distance of 1 mm, which can yield an electric field of approximately 500 kV/m, for 1 minute in place of proteolytic digestion generates high quality ISH results. This step can be completed before deparaffination by standard processes. In some embodiments, methods of exposing DNA comprise applying an electric field at a voltage potential less than 500V, or less than 120V, and/or a voltage potential of less than 12V. The voltage potential applied may be positive or negative, and can be provided by applying a DC or AC voltage to one or more electrode. Exemplary electrodes for use in the present methods include flat electrodes such as a plate and pin electrodes; pairs of electrodes where one or both is stationary or one or both moveable; and electrodes at room temperature, chilled or heated.

The electric field created by the present technology may have a similar effect as proteolytic digestion. The electric field generated does not damage the nuclear material, which is seen with severe pepsin overdigestion. Nucleic acid quality in a tissue sample may be assessed by electrophoresis and quantitative real-time polymerase chain reaction (qRT-PCR) for eukaryotic 18S rRNA. The present methods have been found to avoid or reduce nucleic acid damage and therefore assists exposing the DNA for the assay without nucleic acid damage. In some embodiments, these methods may be performed without producing a corona discharge.

As another aspect, the present technology provides methods of preparing a sample for analysis when the sample comprises one or more polypeptides and the sample has been fixed with an aldehyde such as formaldehyde. In some embodiments, the present methods comprise applying an electric field effective to reverse one or more fixation effects, such as reversing crosslinking by the aldehyde or methylene bridge formation. The methods are especially suitable for tissue samples such as tissue sections, in which the polypeptide comprises one or more antigens to be retrieved. Methods and apparatus are provided for retrieval of antigens in the absence of buffer with a simplistic setup comprising a heat source, paraffin, and an array of electrodes. Applying an electric field to a sample is therefore an improvement over complex and costly antigen retrieval methods, and it avoids loss of tissue sample and improves adhesion of the tissue to the slide. The present methods can eliminate the need for enzymatic digestion of a tissue, as well as the use of buffers, and potentially expensive automated stainers that can be accompanied by a proteolytic digestion step. By using the present technology, the use of buffers and proteolytic digestion steps are not required.

In some embodiments, the present methods comprise moving the electric field a selected number of passes over the sample. In some embodiments, the electric field is not moved. In some embodiments, the electric field generates ozone at the sample. The present methods can also comprise adjusting humidity at the sample.

In some embodiments, the present methods comprise contacting the sample with one or more ionic salts and/or with liquid paraffin, molten paraffin, or other hydrophobic medium having a boiling point above 110°; or above 200°, while applying the electric field. Examples of ionic salts include imidazolinium chloride (C₅H₁₀Cl₂N₂), lithium tetrafluoroborate (LiBF₄), ammonium trifluoracetate (CF₃CO₂NH₄), methyl imidazolium chloride (C₄H₆N₂ HCl), butyl methylimidazolium nitrate (CsHi₅N₃O₃), hexyl methylimidazolium chloride (C₁₀H₁₉ClN₂), 1-ethyl-3-methylimidazolium acetate (C₈H₁₄N₂O₂), and mixtures thereof. For instance, the methods can comprise contacting the sample with a mixture of one or more ionic salts in a hydrophobic medium having a melting point below 50° and a melting point above 110° C. The electric field can be applied for a sufficient time to reverse the fixation effects, for example at least about 5 minutes, or at least about 30 minutes. In some embodiments, the methods also comprise heating the tissue sample at a sufficient temperature to reverse the fixation effects, for example at least about 90° C. or at least about 110° C. By use of the present methods, antigens in the sample can be retrieved without applying an antigen retrieval buffer to the tissue sample.

As another aspect of the present technology, an apparatus is provided for antigen retrieval for samples comprising one or more polypeptides to be analyzed. The present apparatus can include an electric field generating device configured for applying an electric field to a plurality of samples on a plurality of solid substrates. The electric field generating device can comprise a plurality of electrodes comprising a curved feature. The apparatus can also include a holder configured for holding the plurality of solid substrates and for applying heat to the samples; and an actuator configured for moving the plurality of electrodes in a repetitive or predetermined pattern over the solid substrates. In some embodiments, the apparatus also comprises a humidity controller capable of adjusting humidity at the solid substrates. The apparatus can include a housing in which the holder and the electric field generating device are housed, and one or more of a temperature sensor, a humidity sensor, and an ozone sensor inside the housing, and the sensor(s) are in communication with a controller. In some embodiments, the apparatus further comprises a dispenser capable of dispensing a medium having a viscosity of 25 mPa-s or higher, such as liquid paraffin. The apparatus can also comprise a reservoir containing one or more ionic salts, a hydrophobic medium, or mixtures thereof.

In some embodiments, the present methods and apparatus improve adhesion of the tissue (or other sample) to the slide (or other solid substrate), thereby preventing tissue loss during the IHC process. The electric field can generate ozone in an area local to the tissue that may interact with the methylene bridges that cross-link the protein epitopes and nucleic acids during formalin fixation of the tissue, which is the first step in tissue processing. It is believed that the electric field created ozone interacts with humidity in the air to reverse methylene bridges, therefore successfully retrieving epitopes that can then bind to the antibody.

In some embodiments, the present technology allows retrieval of antigens in the absence of aqueous buffer, by applying a medium that contains miscible ionic salts. The antigen retrieval can be accomplished with a setup that includes a heat source, paraffin, and an array of negative electrodes. In some embodiments, the use of electric field will improve complex and costly antigen retrieval methods without loss of tissue sample, as it improves adhesion of the tissue to the slide. In some embodiments, it will eliminate the need for enzymatic digestion of the tissue.

Salt are ionic compounds that can be formed by the neutralization reaction of an acid and a base. Salts are composed of related numbers of cations (positively charged ions) and anions (negative ions) so that the product is electrically neutral (without a net charge). These component ions can be inorganic, such as chloride (Cl⁻), or organic, such as acetate (CH₃CO²⁻), and can be monatomic, such as fluoride (F⁻), or polyatomic, such as sulfate (SO₄²⁻).

Many ionic compounds exhibit significant solubility in water or other polar solvents. Unlike molecular compounds, salts dissociate in solution into anionic and cationic components. The lattice energy, cohesive forces between these ions within a solid, determines the solubility. The solubility is dependent on how well each ion interacts with the solvent.

Salts characteristically have high melting points. Some salts with low lattice energies are liquid at or near room temperature. These include molten salts, which are usually mixtures of salts, and ionic liquids, which usually contain organic cations. These liquids exhibit unusual properties as solvents. The “ionic salts” employed herein are salts that are immiscible in polar solvents and are miscible in non-polar solvents due to hydrophobic chemical moieties. Ionic salts in this context refer to salts with significant solubility in organic hydrocarbons such as liquid paraffin oil and paraffin. In some embodiments of the present methods for antigen retrieval, liquid or molten paraffin containing ionic salts is employed to increase conductivity of electrons in addition to the ionizers used to create an electric field.

As another aspect, the present technology provides methods of analyzing a sample by staining with a plurality of staining reagents such as in multiplex IHC assays. The present methods can comprise staining a sample with a first staining reagent; covering the sample with a flowable cover medium such as paraffin; detecting a first stain pattern of the sample; removing the flowable cover medium from the sample, such as by rinsing with a deparaffinizing solvent such as Clearify, or applying an electric field, or applying a heated air knife; staining the sample with a second staining reagent to form a second stain pattern; and covering the sample with a coverslip or a flowable cover medium. In some embodiments, the sample is a tissue sample comprising one or more polypeptides, and one or both of the first and second staining reagents comprise a primary antibody that specifically finds an antigen of the one or more polypeptides.

For multiplex IHC assays, the use of a flowable cover medium for stabilization and to replace coverslipping after staining with a first staining reagent provides several potential advantages. It can reduce the time required for the multiplex IHC assay by omitting coverslipping step. It can also reduce the amount of image distortion from harsh tissue manipulation. In the present methods, after a first staining procedure a protective layer of paraffin or other flowable cover medium to the tissue section and the slide is heated briefly to drive off prior solvent. Excess paraffin can be removed using either a heated air knife or an electric field for 1 minute at 65° C. The tissue section is then ready for scanning. This can be followed by a brief rinse with a deparaffinizing solvent such as Clearify prior to rehydration for application of a second staining reagent.

The present methods are especially suitable for tissue samples comprising one or more polypeptides, and one or both of the first and second staining reagents can comprise a primary antibody that specifically binds an antigen of the one or more polypeptides. In some embodiments, the methods also comprise performing target retrieval before staining with the first staining reagent. Target retrieval can be performing by applying an electric field (as described by the present disclosure) or by other techniques (such as by proteolytic digestion).

In some embodiments, the first staining reagent comprises a first primary antibody that specifically binds a first antigen; a first secondary antibody that binds the primary antibody; and a first label. The first label (and other labels mentioned herein) can be fluorescent, luminescent, radioactive or chromogenic (for example, chemiluminiscent), or the label can be an enzyme that catalyzes a reaction that produces a chromogenic product or visible product (for example, alkaline phosphatase and horseradish peroxidase, which cleave DAB or BCIP/NBT to produce a brown or purple color). Alternatively, a label can comprise biotin or other moiety that specifically binds another moiety (such as a streptavidin or avidin protein). The labels are generally attached to a secondary antibody or otherwise associated with a secondary antibody. In some embodiments, the second staining reagent comprises a second primary antibody that specifically binds a second antigen. The second staining reagent can comprise a second secondary antibody; and a second label.

Some embodiments of the present methods can include additional steps for an IHC procedure, such as contacting the sample with a protein block agent before the staining with the first staining reagent, wherein the protein block agent blocks non-specific proteins from binding to the first staining reagent. In some embodiments, the first and second staining reagents comprise primary antibodies or nucleic acid probes, and the method further comprises staining the sample with hematoxylin.

In some embodiments, the present methods also comprise denaturing the first primary antibody prior to contacting the sample with the second primary antibody. In some embodiments, the first primary antibody is denatured by contacting with a denaturing agent such as sulfuric acid. In some embodiments, the denaturing is performed prior to the detecting of the first stain pattern. In some embodiments, the first primary antibody is denatured by contacting with a protease. The protease used in the method may be any suitable protease, including a serine protease, a metallo-protease, or a cysteine protease. The amount of protease used in the present methods should be sufficient to proteolytically digest the first and/or second antibodies, while leaving the tissue section and other antigens that are in the sample structurally intact.

In some embodiments, at least one of the first or second staining reagents binds a tumor cell antigen, and at least one of the first or second staining reagents binds an immune cell antigen. The immune cell antigens and the tumor cell antigens can be labeled with stains such as precipitated stains of different colors.

For example, the first primary antibody and the second primary antibody can be antibodies that specifically bind to biomarkers, such as cancer biomarkers. Exemplary cancer biomarkers, include, but are not limited to carcinoembryonic antigen (for identification of adenocarcinomas), cytokeratins (for identification of carcinomas but may also be expressed in some sarcomas), CD15 and CD30 (for Hodgkin's disease), alpha fetoprotein (for yolk sac tumors and hepatocellular carcinoma), CD117 (for gastrointestinal stromal tumors), CD10 (for renal cell carcinoma and acute lymphoblastic leukemia), prostate specific antigen (for prostate cancer), estrogens and progesterone (for tumour identification), CD20 (for identification of B-cell lymphomas) and CD3 (for identification of T-cell lymphomas).

In some embodiments, the label is a fluorophore, such as a fluorophore selected from the group consisting of a coumarin, a cyanine, a benzofuran, a quinoline, a quinazolinone, an indole, a benzazole, a borapolyazaindacene, a xanthene, and combinations thereof. In the present methods of multiplex analysis, fluorophores may be chosen so that they are distinguishable, i.e., independently detectable, from one another, meaning that the labels can be independently detected and measured, even when the labels are mixed. In other words, the amounts of label present (for example, the amount of fluorescence) for each of the labels are separately determinable, even when the labels are co-located (for example, in the same tube or in the same area of the sample).

Examples of fluorescent labels include fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110: cyanine dyes, for example, Cy3, Cy5 and Cy7 dyes; coumarins, for example, umbelliferone; benzimide dyes, for example Hoechst 33258; phenanthridine dyes, for example, Texas Red; ethidium dyes; acridine dyes; carbazole dyes; phenoxazine dyes; porphyrin dyes; polymethine dyes; BODIPY dyes and quinoline dyes.

The staining of samples with fluorescent labels can be viewed with a fluorescence microscope and an appropriate filter for each fluorophore, or by using dual or triple band-pass filter sets to observe multiple fluorophores.

In some embodiments, the present methods can allow a more accurate spatial localization; for example, of immune cells and tumor cells in a tissue section with less tissue distortion and a savings in time from traditional coverslipping methods. The present methods eliminate the need for coverslipping of the tissue prior to whole slide imaging or other analysis. Current process for coverslipping in this workflow for multiplex IHC is harsh and results in tissue distortion. Tissue distortion interferes with the algorithm development process which “maps” that location of cells between whole slide images to teach an algorithm. This method should result in an improvement in the algorithms accuracy.

In some embodiments, the present methods of multiplex IHC detection are used to distinguish immune cells (macrophages, B cells, T cells, and others) from tumor cells in order to derive a more accurate score for the expression of the tumor related protein. For example, the PD-L1 22c3 pharmDx uses an antibody to PD-L1 that stains tumor cells and also immune cells. For scoring purposes, the PD-L1 expression on tumor cells is of importance and staining of immune cells must be omitted. Currently, the scoring method relies on an H&E stained section and a PD-L1 stained section for which the pathologist must review and try to omit immune cells from scoring based on 2 different tissue sections. The ability to label immune cells with a first staining reagent different from labels bound to tumor cells on the same tissue section with a second staining reagent will allow one to distinguish these cells more easily than using a separate H&E stained section. The ability to scan the section after the first staining reagent and again after the second staining reagent allows for more accurate determination of the location of immune cells amongst tumor cells. If the tissue section is coverslipped after the first staining reagent and scanned, the subsequent step that requires removal of the coverslip and mounting media results in distortion of the tissue section for visualization after the second staining reagent is applied. The coverslipping step is very harsh for the tissue section. In addition, removing the coverslips requires the soaking off of mounting media, which adds about 10 minutes to an hour to the process.

As another aspect, the present technology provides methods of preparing a sample for analysis. The methods comprise contacting a sample (such as a tissue section) on a slide (or other solid substrate) with a mounting medium, applying an electric field to the mounting medium for a time sufficient to spread the mounting medium over the sample: and covering the sample on the slide with a coverslip or a flowable cover medium. The present technology allows uniform spreading of the solvent used to wet the tissue prior to application of a mountant, thereby reducing the incidence of artifacts. The application of an electric field will also apply uniform pressure to the coverslip which allows a more precise placement of the coverslip.

In some embodiments, the mounting medium is applied to the coverslip in a plurality of droplets prior to contacting with the sample, such as by spacing the droplets over the area of the coverslip. In some embodiments, the mounting medium is applied to a coverslip, then the coverslip is placed on the sample, then the electric field is applied. The present methods can also comprise applying the electric field to the covered sample so that a static force holds the coverslip in place while the mounting medium is curing. The electric field can be applied for a time sufficient to remove any bubbles between the coverslip and the sample.

Advantages of the present methods include minimizing the volume of mountant used for coverslipping, preventing sticky glue residuals, and eliminating the presence of bubbles by even spreading of wetting solvents such as Clearify. This provides even curing of mounting media across the tissue, and more precise placement of the coverslips, thereby avoiding misalignment. A process that improves the appearance and the morphology of the tissue is beneficial for pathologist evaluation and the tissue section must remain capable of evaluation for many years. The present methods are also beneficial for the ease of handling of the slide for subsequent processes using equipment such as microscopes and scanners, for which glue residuals and misaligned coverslips are undesirable.

In some embodiments, the present methods apply an electric field in a manner that moves solvents such as Clearify and therefore causes even spreading of a thin layer of solvent over a tissue prior to application of mountant and a coverslip. The static field generated by an electrode can also apply pressure to the surface of the coverslip to allow adherence of the coverslip to a slide and prevent the coverslip from moving after placement.

In some embodiments, the present methods evenly spread the solvent across a tissue section or other sample to allow uniform penetration to the tissue section or other sample. A small aliquot (30 μL) of mountant can then be applied to a coverslip or to a slide and tissue section directly and then a glass coverslip can be placed on the slide plus mounting media. An electrode can then be used to apply a static force to the surface of the coverslip to hold it in place as the mountant is curing.

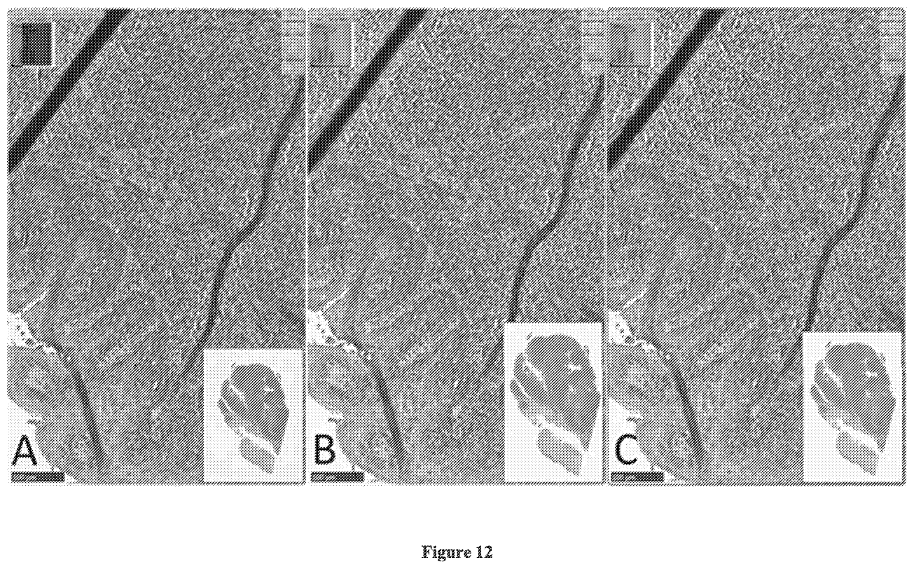

FIG. 13 illustrates an exemplary embodiment of how an electrode at a high potential can generate negative ions. These ions will collect on the surface of the solvent. Surface charged solvent is then repelled by the electric field created from the pin to allow even spreading. A small quantity of mounting media can be applied to the coverslip of the glass slide with stained tissue. The static generated by the pin can then apply force to the surface of the coverslip to assist with application of the coverslip in a precise location.

In some embodiments, the present technology would aim to evenly spread a thin coating of paraffin or other cover medium across the surface of the H&E, IHC or ISH stained tissue section prior to application of a coverslip or in lieu of a coverslip. This thin film would protect the tissue from dry out and oxidation. The process begins by ensuring all solvent from the staining process is completely removed. This is done by applying molten paraffin over the stained tissue; this can be done in various ways such as direct piping of molten paraffin or via a paraffin infused wax sheet. The stained slide is then heated until all the solvent has been evaporated; in some cases, the solvent can be ethanol, xylene, or Histoclear®. Since paraffin has a higher boiling point than solvents used in the staining process, the paraffin will not evaporate. When the solvent evaporates, the sample remains under a layer of paraffin which prevents the stained tissue from being exposed to oxygen. Once all the solvent has evaporated, the slide is processed by applying an electric field in the same way an electric field can remove paraffin from tissue sections. The standard protocol is to place a slide on a 65° C. hot plate and pass a pin charged to a potential of −20 kV over the surface of the slide three times. This process leaves a thin layer of paraffin on top of and embedded within the tissue.

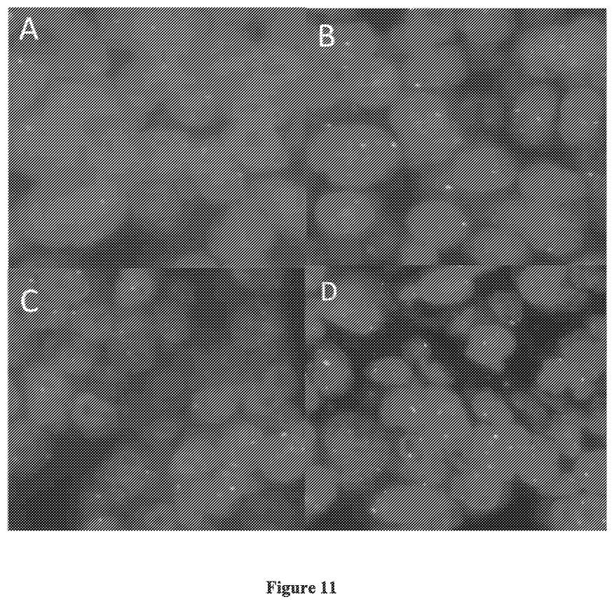

After applying the thin layer of cover medium, the slide is stable and can be coverslipped using any coverslipping technique. In some embodiments, the present methods eliminate the application of additional solvents besides the mounting media. FIG. 12 shows an example of the stability of stained tissue after stabilizing with paraffin.

An alternative to the use of an electric field to evenly spread a thin coating of paraffin across the surface of the stained tissue section is to use a heated air knife to spread the paraffin.

As another aspect, the present technology provides methods of preparing a sample for analysis, comprising contacting a stained sample on a slide with a flowable cover medium such as paraffin; spreading the flowable cover medium over substantially all of the stained sample; and solidifying the flowable cover medium to form a covered coating over the sample. The methods are especially suitable for stained samples comprising a tissue stained with one or more of an immunohistochemical staining reagent, a labeled nucleic acid probe, or hematoxylin and/or eosin. The present technology will allow uniform spreading of a thin coating of paraffin or other flowable cover medium over the tissue section prior to coverslipping or in the absence of a coverslip thereby reducing tissue dry out and oxidation of hematoxylin over time. This thin film is not visible under a coverslip but provides sufficient barrier to limit oxygen diffusion into the tissue.

In some embodiments, the present methods can comprise one or more of the steps of applying an electric field to spread the flowable covering medium; dispensing molten paraffin or other flowable cover medium onto the stained sample; placing a paraffin-infused sheet or cover medium-infused sheet over the stained sample, wherein the flowable cover medium comprises the paraffin, and/or immersing the stained sample in a bath comprising molten paraffin or cover medium. In some embodiments, the present methods also comprise heating the covered sample to remove solvent from the sample while the flowable cover medium prevents exposure of the sample to oxygen. In some embodiments, the present methods also comprise comprising adhering a coverslip to the covered sample. In some embodiments, the present methods also comprise comprising storing the covered sample without a coverslip adhered to the slide. In some embodiments, the sample is stored for at least 1 month, or at least 3 months, or at least six months, or at least one year, or at least five years, or at least 10, 12, 15, 20, 24, or 30 years, without significant loss of staining or signal.

Advantages of some embodiments of the present methods include reducing or eliminating the amount of oxygen exposure of the tissue by placing a thin film of paraffin or other flowable cover medium uniformly over the tissue by applying an electric field. In some embodiments, the present methods improve the appearance and the morphology of the tissue, and make the tissue section stable and evaluable for many years. Some embodiments of the present methods are beneficial for the ease of handling of the slide for subsequent processes using equipment such as microscopes and scanners, as the thin coating is not visible.

The present technology can evenly spread a thin coating of paraffin or other flowable cover medium across the surface of the H&E, IHC or ISH stained tissue section prior to application of a coverslip or in lieu of a coverslip. This thin film would protect the tissue from dry out and oxidation. In some embodiments, the method also comprises removing substantially all solvent from the sample that was added during a staining process.

In some embodiments, molten paraffin or molten cover medium is placed over the stained tissue; this can be done in various ways such as direct piping of molten paraffin or via a paraffin infused wax sheet. The stained sample on the slide is then heated until all the solvent has been evaporated; in some cases, the solvent can be ethanol, xylene, or Histoclear®. As the paraffin will have a higher boiling point than any solvent used in the staining process, the paraffin will not evaporate. When the solvent evaporates, it is under a layer of paraffin preventing the stained tissue from being exposed to oxygen. Once all the solvent has evaporated, the sample is processed by applying an electric field. In some embodiments, a slide is placed on a 65° C. hot plate and an electrode charged to a potential of −20 kV is passed over the surface of the sample or the slide three or more times. This process leaves a thin layer of paraffin on and embedded within the tissue.

As yet another aspect, an apparatus for staining a sample and applying a flowable cover medium is provided. A staining instrument can comprise a final tank that the slide is immersed into, which can be a heated paraffin tank. The slide is immersed in the tank for a time sufficient to evaporate substantially all solvent from the tissue. Then as the slide is removed from the tank, electrodes applying an electric field can be placed above the tank removing the paraffin still bound to the tissue and slide leaving only a residual amount of paraffin. At this point the slide is paraffin stabilized and can be coverslipped or left without coverslip.

From the foregoing, it is evident that the present methods and apparatus for preparing a tissue for analysis may provide a number of advantages. As noted above in the background section of this disclosure, after paraffin-embedded tissue has been sectioned into thin slices, the resulting tissue sections conventionally undergo a number of processes to prepare them for analysis. By comparison, some embodiments of the present tissue preparation device 100 and methods are effective in preparing samples more quickly and with improved quality.

Moreover, in some embodiments, the electric field-based methods of the present disclosure have been found unexpectedly to be not merely compatible with standard hematoxylin and eosin (H&E) staining, but also antigen retrieval and staining for immunohistochemistry (IHC).

Tissue preparation as described herein may conveniently be carried out under ambient conditions. Ambient air may be utilized as a plasma-forming gas mixture. That is, the plasma utilized may be an air plasma. In other embodiments, the plasma-forming process may be enhanced by providing a flow of one or more specific plasma-forming gases to the ionization (plasma-forming) region in the vicinity of the first electrode. Examples of specific plasma-forming gases include, but are not limited to, diatomic oxygen (O₂), diatomic nitrogen (N₂), a noble gas such as argon (Ar), etc. A specific plasma-forming gas may be provided by nozzles configured to direct the gas to the ionization region where the gas can be excited by the applied electric-field. Alternatively, the device 100 and the tissue 108 may be positioned in an enclosure that provides a controlled environment (relative to the ambient), and the plasma-forming gas may be flowed into the enclosure. The use of specific plasma-forming gas(es) to modify the composition of the air in the ionization region, the use of such gas(es) instead of air, may be desired for tuning the conditions (for example, voltage parameters) under which plasma is generated and sustained, for ensuring that the paraffin is ionized preferentially over other components of the tissue 108 that do not need to be ionized, etc.

Thus far, the mechanism or technique of ionization has been described primarily in the context of a corona discharge-type plasma. The presently disclosed subject matter in its broader aspects, however, is not limited to any specific mechanism of ionization. More generally, any type of ionization suitable for the process of paraffin removal may be utilized. Typically, atmospheric-pressure ionization (API) techniques are contemplated as they do not require operating in an evacuated environment. Examples of API techniques include, but are not limited to, atmospheric-pressure plasma-based ionization, atmospheric-pressure chemical ionization (APCI), atmospheric-pressure photoionization (APPI) (for example, using a laser, ultraviolet lamp, etc.), inductively coupled plasma (ICP) ionization, microwave induced plasma (MIP) ionization, dielectric barrier discharge (DBD) plasma ionization, etc. Alternatively, ionization in a vacuum regime may be carried out if the device 100 and the tissue 108 are positioned in an appropriately configured vacuum chamber. Examples ionization techniques performed in vacuum include, but are not limited to, glow discharge (GD) ionization, electron ionization (EI) using a filament for thermionic emission, and chemical ionization (CI) using a filament for thermionic emission.

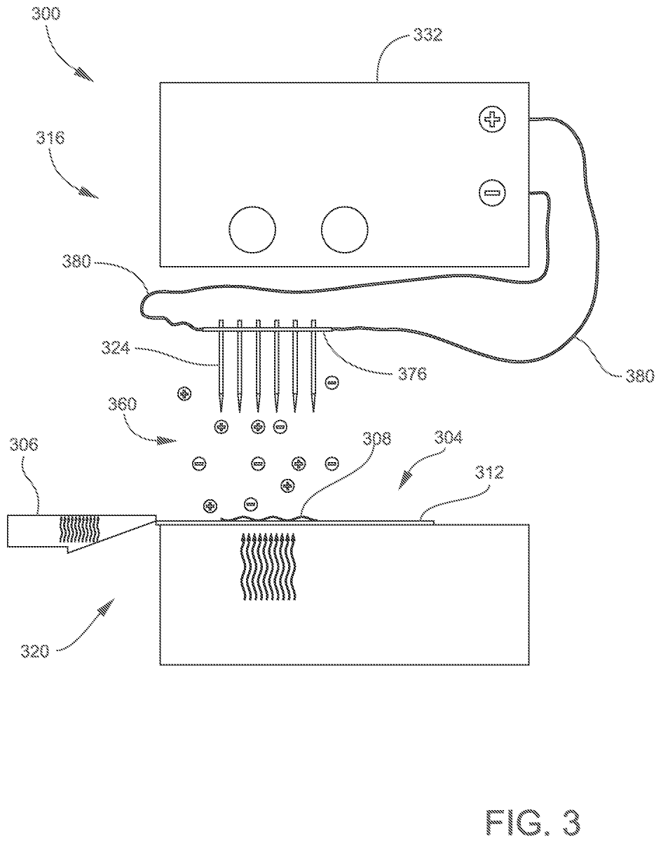

FIG. 3 is a schematic view of a tissue preparation device 300 according to another embodiment. Similar to the embodiment described above and illustrated in FIGS. 1 and 2, the device 300 includes an electric field-generating device 316 and a heating device 320 on which a supported tissue arrangement 304 (paraffin-impregnated tissue 308 on, for example, a microscope slide 312) is supported. In the present embodiment, the electric field-generating device 316 includes a plurality of movable first electrodes 324. The first electrodes 324 may be arranged in a one-dimensional or two-dimensional array. The first electrodes 324 may be spaced from each other by a fixed distance (or pitch). The first electrodes 324 may be (removably) mounted to an appropriately configured electrode support 376 that determines the positions and spacing of the first electrodes 324 relative to each other. In some embodiments, the electrode support 376 may be electrically conductive to minimize the amount of electrical wiring 380 needed between the first electrodes 324 and a voltage source (electrical power supply) 332. The electric field-generating device 316 may further include electrically insulating and/or thermally insulating structures (not shown) attached to or otherwise mechanically referenced to the electrode support 376 and/or the first electrodes 324 as needed to facilitate manual or automated movement of the first electrodes 324 across the tissue 308. For example, electrically insulating and/or thermally insulating structures may be provided in the form of a user-graspable handle or a fixture configured for coupling to a motorized stage or robot. Also in the illustrated example, the voltage source 332 is provided in a control console that includes user-operated control knobs for adjusting voltage, or both voltage and frequency in the case of an AC voltage source.

The multiple first electrodes 324 may be useful for increasing the overall size or footprint of the active region of plasma 360 generated by the electric field-generating device 316. In this case, the plasma 360 may be shaped as a wide curtain. The device 300 may otherwise generally be configured and operated similarly to the device 100 described above and illustrated in FIGS. 1 and 2.

One or more second electrodes (not specifically shown) utilized as a counter-electrode or ground electrode may be disposed on or integrated with the heating device 320, or otherwise positioned proximate to the tissue 308.

As further shown in FIG. 3, the device 300 (or any of the other embodiments of the tissue preparation device disclosed herein) may include a paraffin application device 306 utilized to apply paraffin to the tissue 308. The paraffin application device 306 can be used to apply paraffin or other flowable cover medium as an aid to coverslipping or instead of a coverslip. The paraffin application device 306 may be movable, or its position adjustable, relative to the heating surface of the (main) heating device 320. In some embodiments, the paraffin application device 306 may be composed of a thermally conductive material and in operation may be heated to assist in providing a flowable medium.

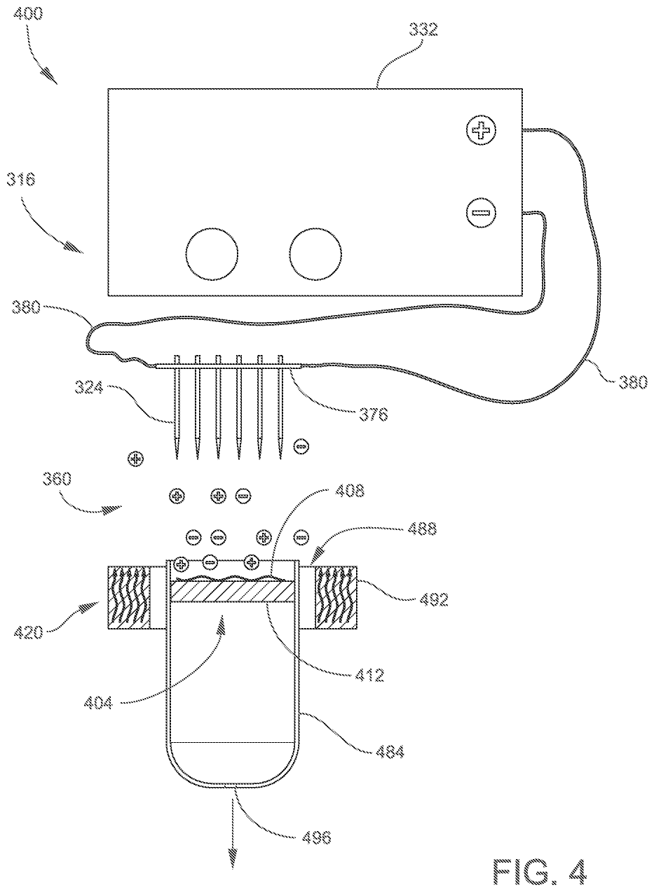

FIG. 4 is a schematic view of a tissue preparation device 400 according to another embodiment. Similar to other embodiments described herein, the device 400 includes an electric field-generating device 316 and a heating device 420 near which a supported tissue arrangement 404 is positioned. By way of example, the electric field-generating device 316 with multiple first electrodes 324 described above is also provided in the present embodiment, although any of the other electric field-generating devices described herein may be included in the tissue preparation device 400. In the present embodiment, a paraffin-impregnated tissue 408 is supported on a solid substrate in the form of a porous frit 412, which may be made of glass or other material capable of withstanding the heat applied during tissue removal. The frit 412 may be removably mounted in a liquid container 484 (for example, column, vial, test tube, vessel, flask, well of a microtiter plate, etc.) such as at or near the open top of the container 484. The container 484 may be removably mounted in an opening 488 of a container support 492 (for example, a support plate with one or more openings). The container support 492 may include an array of openings 488 for supporting respective containers 484 and corresponding tissues 408 and frits 412.

In the present embodiment, all or part of the heating device 420 may be integrated with the container support 492. For example, the heating device 420 may include a plurality of circumferentially spaced (relative to the longitudinal axis of the container support 492) heating elements positioned on or in the container support 492 in close enough proximity to the tissue sample 404 to be in thermal contact with the tissue 408. In the present context, the term “in thermal contact” generally may be taken to mean that the heating elements are positioned close enough to the tissue 408 such that when they are activated they establish a thermal gradient effective for transferring an amount of heat to the tissue 408 within a short period of time, for example within a minute or a few minutes.

One or more second electrodes (not specifically shown) utilized as a counter-electrode or ground electrode may be disposed on or integrated with the heating device 420, or otherwise positioned proximate to the tissue 408.

In the present embodiment, solvents or paraffin may drain through the holes or pores of the frit 412 and be collected in the container 484. In some embodiments, the container 484 may include an opening 496 at its base to allow solvents or paraffin to drain out from the container 484.

In some embodiments, the sample may be disposed directly on a surface of a liquid container (for example, column, vial, test tube, vessel, flask, well of a microtiter plate, etc.), in which case the liquid container serves as the solid support for the sample.

In some embodiments described herein, the sample (and underlying substrate, if provided) are depicted or described as being horizontally or vertically oriented. However, the sample (and underlying substrate, if provided) may be oriented vertically or horizontally instead (unless a particular orientation is specifically recited) or at any angle between the horizontal and vertical reference planes (in a range from zero degrees to ninety degrees relative to a horizontal reference plane). By way of example, orienting the sample at an angle to the horizontal may enhance water removal through the assistance of gravity.

Various other embodiments of a sample preparation device encompassed by the present disclosure may include combinations of features from different embodiments described above. Various embodiments of the methods encompassed by the present disclosure may include combinations of features from different embodiments described above.

### EXAMPLES

### Example 1

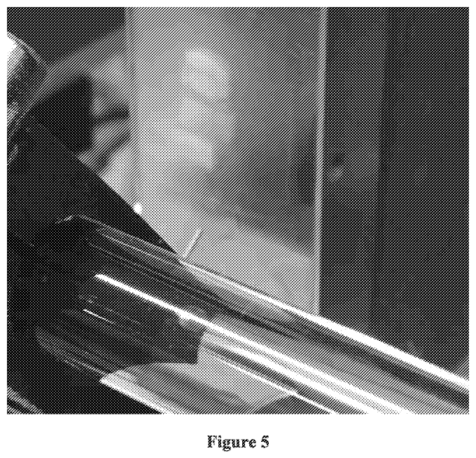

This example demonstrates removal of water trapped between a sample (more particularly, a tissue section) and a solid substrate (more particularly, a slide) using an electric field. A first electrode (more particularly, a pin) was charged with −20 kV, thereby creating a corona discharge. The pin was sufficiently close to the tissue section so that an electric field was applied, causing trapped water to move downward. As shown in FIG. 5, water began to pool underneath the pin, as water was pulled from between the tissue and slide. The location that the water tends to pull from depends on where the arc to ground point is.

Once the water pooled at the pin is large enough, gravity will cause the droplet to fall, speeding up the drying time. It is not necessary for the droplet to fall, but this improves drying performance. The path that the water drains from depends on where the arc to ground points are. For example, in FIG. 5, the arc points were on the left side of the slide, at both the top and bottom of the tissue, which prevented the water trapped on the top right from draining efficiently. Accordingly, in some embodiments, the arc is adjusted to increase water removal.

Initial testing included 3 minutes of exposure to this electric field drying method. After drying was complete, the temperature on the block was increased to melt the paraffin, and an electric field can be used to deparaffinize the section and adhere the tissue. Alternatively, the section may be deparaffinized using standard processes. This deparaffinization process allowed the observation of qualitative results of drying efficiency. Of five tests, only one had residual water remaining on the slide. That tissue had residual water in a region where no arc paths to ground existed.

Another configuration for applying the electric field would have the pin to the right of the tissue and the arc to ground point on the left. The pin would start at the top of the tissue, making its way down the slide. This would dry out the tissue starting from the top moving down, ensuring the full area of the tissue is dry in a more consistent manner. Other ground and pin configurations include both electrodes as pins, which work to some extent but not as well as the ground plate and pin combination.

### Example 2

In this example, the use of an electric field for antigen recovery was examined in the context of an IHC protocol for staining Vimentin in tissue sections of breast carcinoma. Vimentin is a filament protein express in mesenchymal cells.

Tissue sections were stained using the DAKO Omnis automated staining platform. That platform is capable of performing HIER on individual tissue sections using DAKO Target Retrieval Solution (a Tris/EDTA high pH buffer solution). In this example, for the experimental tissue sections, the HIER step of the DAKO Omnis platform was omitted from the IHC protocol. Instead, for the experimental tissue sections, an electric field was applied at 110° C. for 30 minutes, with a negatively charged pin constantly moving over the surface of the tissue sections.

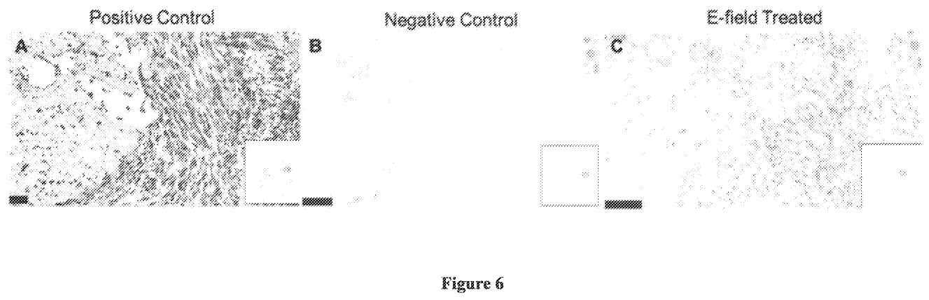

Results for this experiment are shown in FIG. 6. IHC was performed using the DAKO Omnis automated staining platform with HIER (A) or with application of an electric field at 110° C. for 30 minutes in place of HIER (C). Expression of vimentin (brown DAB staining) is seen in stromal cells of the positive control tissue and Electric field treated tissue sections. A negative control (B) shows little or no expression of vimentin. FIG. 6 is at 100× magnification. In the experimental tissue sections, positive expression of vimentin in visible as a result of the IHC protocol. This demonstrates that antigen recovery can be successfully performed by applying an electric field, and without contacting the sample with an antigen recovery buffer.

### Example 3

In this example, the use of an electric field with ionic salts for antigen recovery was examined. A vimentin IHC protocol was used to assess the effect of electric field treatment (Electric field) with added ionic salts on heat induced epitope retrieval. Tissue sections were compared by staining using the DAKO Omnis automated staining platform which performs HIER on individual tissue sections using DAKO Target Retrieval Solution that is a Tris/EDTA high pH buffer solution. For the experimental tissue sections, the HIER step was omitted from the standard IHC protocol. Instead, an electric field was applied to the experimental tissue sections at 95° C. for 5 minutes with the negatively charged pin constantly moving over the surface.

Immunostaining of colon carcinoma for vimentin using electric field treatment and ionic salt paraffin solutions or a standard buffer-based HIER method. IHC was performed using the DAKO Omnis automated staining platform with HIER at 97° C. for 30 minutes (A) or using electric field treatment at 95° C. for 5 minutes in place of HIER (B). 10 mM ionic salt formulations dissolved in paraffin oil (C—H) were electric field treated at 95° C. for 5 minutes for comparison the electric field treatment for 5 minutes at 95° C. alone (B). The ionic salts used in this experiment are (C) Imidazolinium chloride (C₅H₁₀Cl₂N₂), (D) Lithium tetrafluoroborate (LiBF₄), (E) Ammonium trifluoracetate (CF₃CO₂NH₄), (F) Methyl imidazolium chloride (C₄H₆N₂ HCl), (G) Butyl methylimidazolium nitrate (C₈H₁₅N₃O₃) and (H) Hexyl methylimidazolium chloride (C₁₀H₁₉ClN₂). Expression of vimentin (brown DAB staining) is seen in stromal cells of the positive control tissue, Electric field treated tissue sections coated in ionic salt paraffin solution. The level of expression of vimentin increased compared to the electric field treated control (B) for ionic salt paraffin coated tissues (C—H).

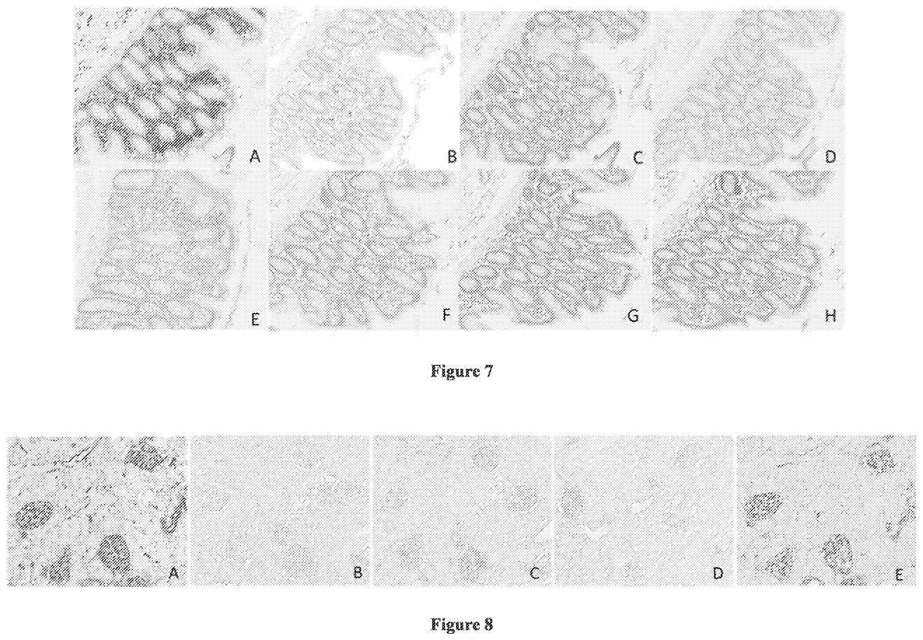

FIG. 7 shows the stained samples at 100× magnification. The experimental tissue sections of colon carcinoma show positive expression of vimentin by IHC. The addition of ionic salts in liquid paraffin solutions with electric field treatment resulted increased staining intensity for vimentin as compared to Electric field treatment alone.

In this example, parameters for antigen retrieval; heat, ionic salt and electric field treatment were further tested. A comparison was made between exposure to high heat (110° C.) for 2 hours (as done in the standard antigen retrieval used by the DAKO Omnis platform), compared to 2 hours of electric field treatment at high heat. Electric field treatment showed higher levels of antigen retrieval, compared to high heat alone. A comparison was also made between the addition of a 1:1 ionic salt/liquid paraffin solution to tissue with high heat alone or with electric field treatment for 30 minutes. FIG. 8 shows that very little antigen retrieval was seen in the high heat-treated tissue with or without the addition of ionic salts. Antigen retrieval was highest for tissue sections that were prepared by applying high heat and an electric field for a 30-minute treatment. This indicates that there is an added benefit of ionic salt and Electric field treatment for antigen retrieval. The use of an ionic salt and liquid paraffin mixture for 30 minutes was more effective than applying an electric field treatment for 2 hours, demonstrating that ionic salt decreased the amount of time required for antigen retrieval compared to an electric field alone.

Results are shown in FIG. 8, which is imaged using a 10× objective (Original magnification, 100×). Immunostaining of kidney tissue for vimentin using standard buffer-based HIER compared to variations in heat, time and addition of a 1:1 ionic salt:liquid paraffin mixture with Electric field treatment. IHC was performed using the DAKO Omnis automated staining platform with HIER at 97° C. for 30 minutes (panel A) or using an oven at 110° C. for 2 hours (panel B), Electric field treatment at 110° C. for 2 hours (panel C), 35 minutes with ionic salt/liquid paraffin mixture in an oven (panel D) or Electric field treatment at 110° C. for 30 minutes with ionic salt/liquid paraffin (panel E). The ionic salt 1-Ethyl-3-methylimidazolium acetate (C₈H₁₄N₂O₂) was mixed in a 1:1 ratio with liquid paraffin oil. Expression of vimentin (brown DAB staining) is seen in glomeruli of the positive control and treated tissues. The level of expression of vimentin increased the most with a 30 minute ionic salt:liquid paraffin mixture electric field treatment at 110° C. FIG. 8.

### Example 4

In this example, the effect of applying an electric field in the preparation of a sample for in situ hybridization was assessed. This example compared samples treated with an electric field to samples treated by proteolytic digestion with pepsin. The preparations were done on sections of breast tissue, which were deparaffinized using three 5-minute rinses of xylene or by applying an electric field. The tissue sections were hybridized according to a standard FISH protocol HER2 and NEAT1 RNA FRIGG FISH assays. HER2 amplification is frequently seen in breast carcinoma and can be a useful biomarker for therapy with Herceptin. NEAT1 is a nuclear RNA, localized to discrete foci called paraspeckles. The standard FISH protocol included a 5-minute pepsin digestion; for the experimental samples (those which had an electric field applied), this pepsin digestion step was omitted.

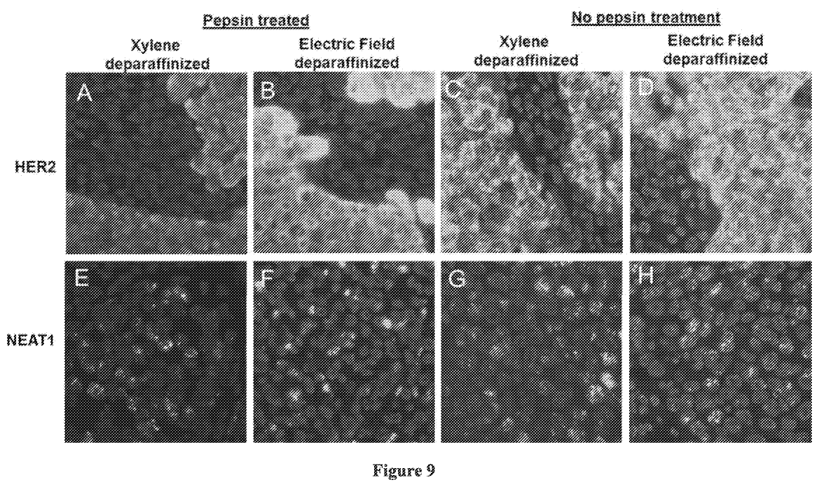

Results of the FISH assays are shown in FIG. 9, which is imaged using a 60× objective (Original magnification, 600×). HER2 FRIGG FISH was performed in tissues with pepsin treatment (panels A & B) or no pepsin treatment (panels C & D). NEAT 1 FRIGG FISH was performed in tissues with pepsin treatment (panels E & F) and without pepsin treatment (panels G & H) for xylene deparaffinized tissue samples (panels A, E, C & G) or electric field treated tissue samples (panels B, F, D & H). In panels B and F, the nuclei of the cells within the samples (DAPI labelled; blue) show undesirable nuclear artifacts or ghost cells, which is due to overdigestion from using both the electric field and pepsin treatment. HER2 (FITC; green) looked equivalent in the xylene deparaffinized, pepsin treated control sample (panel A) and the electric field treated tissues without pepsin (panel D). NEAT1 (FITC; green) signal was brighter and had less background (better signal to noise ratio) in the Electric field treated tissue without pepsin treatment (panel H) than in the xylene deparaffinized pepsin treated control (panel E).

Undesirable nuclear artifacts were seen with the combination of pepsin treatment and electric field treatment, but those artifacts were absent in the experimental tissue (prepared with an electric field but without pepsin). HER2 FRIGG FISH in breast carcinoma controls deparaffinized with xylene including pepsin treatment was equivalent to electric field treated breast carcinoma without pepsin treatment indicating the lack of need for proteolytic digestion with electric field. NEAT1 FRIGG FISH in the breast tissue prepared by applying an electric field showed a brighter signal with less background or a better signal to noise ratio compared to the xylene-deparaffinized pepsin-treated control. These results indicate that electric field treatment is sufficient for probe penetration into the cytoplasm in FISH and may improve probe signal and background for some assays. A brighter probe signal with the elimination of background increases the ease of quantitation of signal. The elimination of nuclear artifacts by the present methods is shown, and there was also a reduction in the time required to perform the assays with electric field use.

### Example 5

In this example, a DNA FISH assay was performed with the addition of a brief rinse of Clearify™ to remove residual paraffin following treatment with an electric field.

FIG. 10 shows the in situ hybridization of breast carcinoma using DNA probes to HER2/CEN17. HER2/CEN17 FISH was performed in xylene deparaffinized tissues with pepsin treatment (panel A) or no pepsin treatment (panel B). HER2/CEN17 FISH was performed in electric field treated and Clearify™ deparaffinized tissues without pepsin digestion (panel C). The traditional workflow without pepsin digestion step results in unacceptable results for FISH, as shown in panel B, as compared to the control (panel A), with an absence of probe signal and poor morphology. Electric field treatment combined with a brief Clearify™ rinse resulted in FISH results comparable to the control, with an absence of undesirable nuclear artifacts or ghost cells due to pepsin overdigestion. HER2 (CY3; red) probe signal looked equivalent in the xylene deparaffinized, pepsin treated control (A) and electric field deparaffinized tissues without pepsin (panel C). CEN17 (FITC; green) signal was equivalent in the controls compared to Electric field treated tissue without pepsin digestion. The ratio of HER2/CEN17 was an average of 1.52 (n=2 controls) as compared to 1.55 (n=3 experimental tissues), indicating equivalent results. FIG. 10 imaged using a 100× objective (Original magnification, 1000×).

The HER2/CEN17 FISH results were equivalent between the control samples and the experimental samples (those treated by applying an electric field) with regards to morphology and probe binding. A pathologist scoring of the ratio of HER2 to CEN17 resulted in equivalent results (HER2/CEN17 ratio for controls was 1.52, versus 1.55 for the experimental tissues treated with an electric field). The brief Clearify™ rinse in combination with application of an electric field facilitated probe binding and eliminated the presence of nuclear artifacts. This example indicates that Clearify™ or similar solvents can be used in combination with the application of an electric field to prepare samples for in situ hybridization.

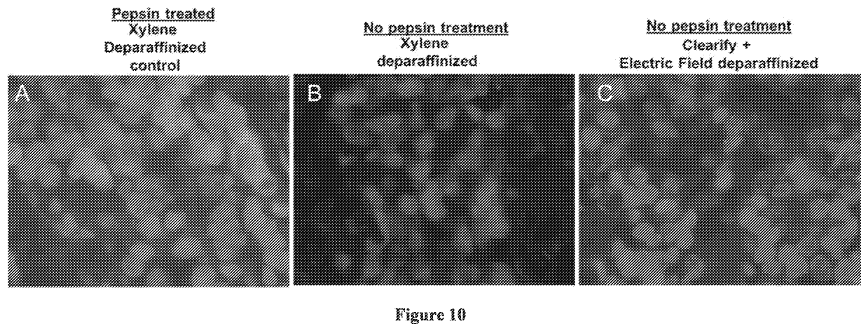

FIG. 11 shows the FISH HER2/CEN17 results for two patient samples of breast adenocarcinoma treated with pepsin (panels A and C) or an electric field (panels B and D). The nuclear morphology as indicated by DAPI stain is good for the pepsin treated control in (A) and sub-optimal for the pepsin treated control in (C). The HER2 (red) and CEN17 (green) probe signals are good in pepsin treated control (A) but HER2 probe signal was absent in pepsin treated control (C). Electric field treated tissue samples (B and D) both have good nuclear morphology and brighter more abundant HER2 and CEN17 probe signals. This data indicates that Electric field treatment may be superior to enzymatic digestion by pepsin for FISH. FIG. 11 images are an inset of photos that were imaged using a 100× objective (Original magnification, 1000×).

### Example 6

In this example, the use of paraffin as a flowable cover medium was evaluated. A breast tissue sample was stained, then flowable paraffin was used to completely cover the sample on Day 0. Heat was applied to remove all liquid from the tissue sample while leaving the paraffin intact. To remove excess paraffin, the slide with sample was placed on a grounded hot plate while a pin charged to −20 kV was passed over the sample three times at a distance of 5 mm. These passes removed excess paraffin, leaving only a residual amount left on the tissue of the sample. The slide was placed in a 65° C. oven on Day 3 and images of the tissue were taken on A) Day 5, B) Day 11, and C) Day 14. This heating simulates accelerated aging. FIG. 11 shows the stained tissue after stabilizing with paraffin.

Over the two-week period, which simulates roughly 3 months of aging, the staining on the tissue did not changed. Under normal circumstances if a slide were left out in the absence of a solvent to keep the tissue wet, the tissue would start to have visible signs of dry out which are indicated by dark nuclei. This example shows that a flowable cover medium successfully stabilized the sample, preserving its staining from loss or instability due to heat.

### Example 7

In this example, a H&E stained tissue section was coverslipped with the aid of an electric field. A solvent (Clearify) was spread evenly using an electric field. To remove excess Clearify, the slide with sample was placed on a grounded plate at room temperature while a pin charged to −20 kV was passed over the sample three times at a distance of 5 mm. These passes removed excess Clearify, leaving only a residual amount left on the tissue of the sample. 30 ul of mounting media was applied to the coverslip as in FIG. 13. The coverslip was placed onto the slide and tissue section. An electric field was then applied to assist in adherence of the coverslip to the slide. The field was created with a pin at a 5 mm distance from the slide and coverslip. The pin was charged to −20 kV and passed back and forth for 3 minutes while the mountant was cured using UV light.

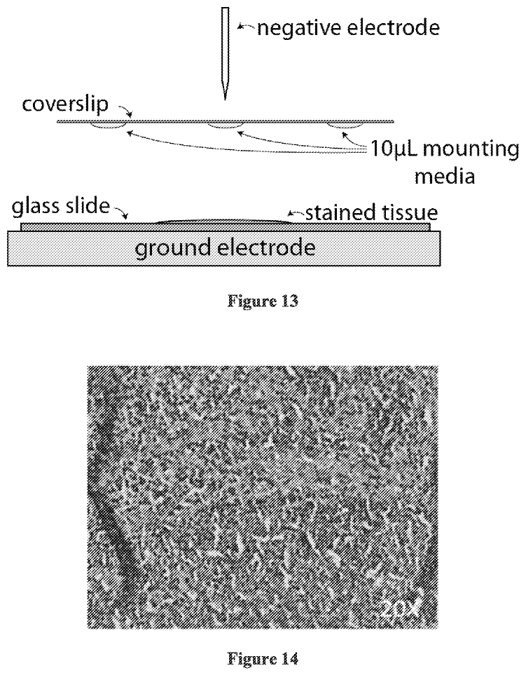

FIG. 14 shows the coverslipped sample slide. There are no bubbles or tissue drying artifacts present, indicating that the coverslip was adhered to the sample without causing distortions.

### EXEMPLARY EMBODIMENTS

Exemplary embodiments provided in accordance with the presently disclosed subject matter include, but are not limited to, the following:

Embodiment 1. A method of drying a tissue sample comprising:
applying an electric field to a tissue sample on a solid substrate having water between the tissue sample and the solid substrate, wherein the electric field is applied at a strength and for a time sufficient to cause at least a portion of the water to move towards a first electrode.

Embodiment 2. The method of embodiment 1, wherein substantially all of the water moves toward the first electrode.

Embodiment 3. The method of embodiment 1 or 2, further comprising:
placing the tissue sample comprising an embedded tissue in a liquid bath;
contacting the embedded tissue with the solid substrate so that the tissue is spread on a surface of the solid substrate; and
removing liquid trapped between the substrate surface and the sample by applying the electric field to the tissue sample.

Embodiment 4. The method of any of embodiments 1 to 3, wherein the solid substrate is a slide having first and second slide surfaces.

Embodiment 5. The method of any of embodiments 1 to 4, further comprising moving the slide so that the slide surface is substantially vertical when the trapped liquid is removed.

Embodiment 6. The method of any of embodiments 1 to 5, wherein applying the electric field comprises applying a voltage between the first electrode and a second electrode.

Embodiment 7. The method of embodiment 6, wherein applying the electric field produces a corona discharge in a region surrounding the first electrode, and further comprising positioning the first electrode so as to expose the trapped liquid to the corona discharge.

Embodiment 8. The method of embodiment 6 or 7, wherein the slide surface is substantially vertical so that the tissue has a top edge and a bottom edge, and the first electrode is located below the bottom edge.

Embodiment 9. The method of any of embodiments 6 to 8, further comprising moving the first electrode from the top edge to the bottom edge while applying the electric field, so that the trapped liquid moves toward the bottom edge.

Embodiment 10. The method of embodiment 9, further comprising returning the first electrode to a position at the top edge and repeating the movement of the first electrode to the bottom edge while applying the electric field.

Embodiment 11. The method of any of embodiments 6 to 10, wherein the voltage is at least 5 kV.

Embodiment 12. The method of any of embodiments 6 to 10, wherein the voltage is at least 10 kV.

Embodiment 13. The method of any of embodiments 6 to 10, wherein the voltage is at least 20 kV.

Embodiment 14. The method of any of embodiments 6 to 13, wherein the first electrode is between about 1 mm and about 7 mm away from the tissue.

Embodiment 15. The method of any of embodiments 6 to 13, wherein the first electrode is between about 3 mm and about 5 mm.

Embodiment 16. The method of any of embodiments 6 to 15, wherein the tissue contacts a first slide surface, and the first electrode is positioned facing a second slide surface.

Embodiment 17. The method of any of embodiments 6 to 16, wherein the tissue contacts a first slide surface, and wherein the second electrode is in contact with a second slide surface, and the second electrode is at a temperature below the melting point of paraffin.

Embodiment 18. A method of preparing a sample for in situ hybridization, the method comprising:
applying an electric field to a sample comprising a target polynucleotide; and
contacting the sample with one or more detectable nucleic acid probes which specifically bind to the target polynucleotide while the electric field is being applied.

Embodiment 19. The method of embodiment 18, wherein the sample is a paraffin-embedded tissue specimen.

Embodiment 20. The method of embodiment 18 or 19, wherein the sample is a whole tissue.

Embodiment 21. The method of any of embodiments 18 to 20, wherein the sample comprises a slice of whole tissue.

Embodiment 22. The method of any of embodiments 18 to 20, wherein the sample is one or more cell pellets.

Embodiment 23. The method of any of embodiments 18 to 20, wherein the sample is one or more cell smears.

Embodiment 24. The method of any of embodiments 18 to 23, wherein the sample comprises one or more cells and the target polynucleotide is within a nucleus of the cells.

Embodiment 25. The method of any of embodiments 18 to 24, wherein the electric field is applied at a strength and for a time sufficient to expose the DNA.

Embodiment 26. The method of embodiment 25, wherein the electric field is applied at a potential of 500V or less.

Embodiment 27. The method of any of embodiments 18 to 26, wherein the sample is not contacted with a proteolytic enzyme before the contacting with the nucleic and probe.

Embodiment 28. The method of any of embodiments 18 to 27, wherein the sample is substantially free of autofluorescence from intact proteins during analysis of the labeled probes hybridized to the polynucleotide.

Embodiment 29. The method of any of embodiments 18 to 28, further comprising removing substantially all paraffin from the sample before the contacting with the labeled probes.

Embodiment 30. The method of any of embodiments 18 to 29, wherein the paraffin is removed by a combination of applying an electric field to the sample and rinsing the sample with a deparaffinization solvent.

Embodiment 31. A method of preparing a sample for analysis, wherein the sample comprises one or more fixed polypeptides, the method comprising:
applying an electric field effective to reverse at least a portion of one or more fixation effects.

Embodiment 32. The method of embodiment 31, wherein the sample has been fixed with an aldehyde, and the one or more fixation effects comprise crosslinking by an aldehyde or methylene bridge formation.

Embodiment 33. The method of embodiment 31 or 32, wherein the sample is a tissue sample, and the polypeptide comprises one or more antigens.

Embodiment 34. The method of any of embodiments 31 to 33, wherein the tissue sample is a tissue section.

Embodiment 35. The method of any of embodiments 31 to 34, wherein the electric field generates ozone at the sample.

Embodiment 36. The method of any of embodiments 31 to 36, further comprising adjusting humidity at the sample.

Embodiment 37. The method of any of embodiments 31 to 36, further comprising contacting the sample with one more ionic salts while applying the electric field.

Embodiment 38. The method of embodiment 37, wherein the ionic salt is selected from the group consisting of imidazolinium chloride (C₅H₁₀Cl₂N₂), lithium tetrafluoroborate (LiBF₄), ammonium trifluoracetate (CF₃CO₂NH₄), methyl imidazolium chloride (C₄H₆N₂ HCl), butyl methylimidazolium nitrate (C₈H₁₅N₃O₃) and hexyl methylimidazolium chloride (C₁₀H₁₉ClN₂), 1-ethyl-3-methylimidazolium acetate (C₈H₁₄N₂O₂), and mixtures thereof.

Embodiment 39. The method of any of embodiments 31 to 38, wherein the sample is embedded in a medium selected from the group consisting of paraffin and hydrophobic embedding medium, and the method further comprises melting the medium by applying an electric field.

Embodiment 40. The method of embodiment 39, wherein the medium has a boiling point the medium has a boiling point above 110° C.

Embodiment 41. The method of embodiment 39, wherein the medium has a boiling point above 200° C.

Embodiment 42. The method of any of embodiments 31 to 41, further comprising contacting the sample with a mixture of one or more ionic salts in a hydrophobic medium having a melting point below 200° C. and a melting point above 110° C.

Embodiment 43. The method of any of embodiments 31 to 42, wherein the electric field is applied for a sufficient time and at a sufficient strength to reverse at least a portion of the fixation effects.

Embodiment 44. The method of embodiment 43, herein the electric field is applied for at least about 5 minutes.

Embodiment 45. The method of embodiment 43, herein the electric field is applied for about 30 minutes.

Embodiment 46. The method of any of embodiments 31 to 45, further comprising heating the tissue sample at a sufficient temperature to reverse the fixation effects.

Embodiment 47. The method of embodiment 46, wherein the tissue sample is heated to a temperature of at least about 90° C.

Embodiment 48. The method of embodiment 46, wherein the tissue sample is heated to a temperature of at least about 110° C.

Embodiment 49. The method of any of embodiments 31 to 48, wherein antigens in the sample are retrieved without applying a buffer to the tissue sample.

Embodiment 50. An apparatus for applying an electric field to a tissue sample, the apparatus comprising:
an electric field generating device configured for applying an electric field to a plurality of tissue samples on a plurality of solid substrates, the device comprising a plurality of electrodes comprising a curved feature;
a holder configured for holding the plurality of solid substrates and for applying heat to the issue samples; and
an actuator configured for moving the plurality of electrodes in a repetitive or predetermined pattern over the solid substrates.

Embodiment 51. The apparatus of embodiment 50, further comprising a humidity controller capable of adjusting humidity at the solid substrates.

Embodiment 52. The apparatus of embodiment 50 or 51, further comprising one or more of a temperature sensor, a humidity sensor, and an oxone sensor inside the housing.

Embodiment 53. The apparatus of embodiment 52, wherein the sensor(s) are in communication with a controller.

Embodiment 54. The apparatus of any of embodiments 50 to 53, further comprising a dispenser capable of dispensing a liquid paraffin.

Embodiment 55. The apparatus of embodiment 54, further comprising a reservoir containing one or more ionic salts, a hydrophobic medium, or mixtures thereof.

Embodiment 56. A method of analyzing a sample by staining with a plurality of staining reagents, the method comprising:
staining a sample with a first staining reagent,
covering the sample which has been stained with the first staining reagent with a flowable cover medium;
detecting a first stain pattern of the sample;
removing the flowable cover medium from the sample;
staining the sample with a second staining reagent to form a second stain pattern; and
covering the sample with a coverslip or a flowable cover medium.

Embodiment 57. The method of embodiment 56, wherein the flowable cover medium is paraffin.

Embodiment 58. The method of embodiment 56 or 57, wherein the flowable cover medium is removed by rinsing with a deparaffinizing solvent.

Embodiment 59. The method of embodiment 56 or 57, wherein the flowable cover medium is removed by applying an electric field.

Embodiment 60. The method of embodiment 56 or 57, wherein the flowable cover medium is removed by applying a heated air knife.

Embodiment 61. The method of any of embodiments 56 to 60, wherein the sample is a tissue sample comprising one or more polypeptides, and one or both of the first and second staining reagents comprise a primary antibody that specifically binds an antigen of the one or more polypeptides.

Embodiment 62. The method of any of embodiments 56 to 61, wherein the sample is a tissue sample comprising one or more polypeptides and one or both of the first and second staining reagents comprise a secondary antibody that specifically binds a primary antibody.

Embodiment 63. The method of any of embodiments 56 to 62, further comprising performing target retrieval before staining with the first staining reagent, wherein the target retrieval is performing by applying an electric field.

Embodiment 64. The method of any of embodiments 56 to 63, wherein the first staining reagent comprises a first primary antibody that specifically binds a first antigen; a first secondary antibody that binds the primary antibody; and a first label.

Embodiment 65. The method of any of embodiments 56 to 64, wherein the second staining reagent comprises a second primary antibody that specifically binds a second antigen.

Embodiment 66. The method of embodiment 65, wherein the second staining reagent further comprises a second secondary antibody; and a second label.

Embodiment 67. The method of any of embodiments 56 to 66, wherein at least one of the first or second staining reagents binds a tumor cell antigen, and at least one of the first or second staining reagents binds an immune cell antigen.

Embodiment 68. The method of embodiment 67, further comprising staining the immune cell antigens and the tumor cell antigens with stains.

Embodiment 69. The method of embodiment 67, further comprising staining the immune cell antigens and the tumor cell antigens with precipitated stains of different colors.

Embodiment 70. The method of any of embodiments 56 to 69, further comprising contacting the sample with a protein blocking agent before the staining with the first staining reagent, wherein the protein block agent blocks non-specific proteins from binding to the first staining reagent.

Embodiment 71. The method of any of embodiments 56 to 70, wherein the first and second staining reagents comprise primary antibodies, nucleic acid probes or both, and the method further comprises staining the sample with hematoxylin.

Embodiment 72. The method of embodiment 71, further comprising denaturing the first primary antibody prior to contacting the sample with the second primary antibody.

Embodiment 73. The method of embodiment 72, wherein the first primary antibody is denatured by contacting with a denaturing agent.

Embodiment 74. The method of embodiment 73, wherein the denaturing agent comprises sulfuric acid.

Embodiment 75. The method of embodiment 73, wherein the denaturing is performed prior to the detecting of the first stain pattern.

Embodiment 76. A method of preparing a sample for analysis, the method comprising:
contacting a sample on a slide with a solvent that reacts with a mounting medium;
applying an electric field to for a time and at a strength sufficient to spread the solvent over the sample;
contacting the solvent with the mounting medium; and
covering the sample on the slide with a coverslip or a flowable cover medium.

Embodiment 77. The method of embodiment 76, wherein the sample is a tissue section.

Embodiment 78. The method of embodiment 76 or 77, further comprising applying the mounting medium to the coverslip in a plurality of droplets prior to contacting with the sample.

Embodiment 79. The method of embodiment 78, wherein the droplets are spaced over the area of the coverslip.

Embodiment 80. The method of any of embodiments 76 to 79, wherein the mounting medium is applied to a coverslip, then the coverslip is placed on the sample.

Embodiment 81. The method of any of embodiments 76 to 80, further comprising applying an electric field to the covered sample so that a static force holds the coverslip in place while the mounting medium is curing.

Embodiment 82. The method of any of embodiments 76 to 81, wherein the electric field is applied for a time and at a strength sufficient to remove any bubbles between the coverslip and the sample.

Embodiment 83. A method of preparing a sample for analysis, comprising:
contacting a stained sample on a slide with a flowable cover medium;
applying an electric field to the stained sample for a time and at a strength sufficient to spread the flowable cover medium over substantially all of the stained sample; and
solidifying the flowable cover medium to form a covered coating over the sample.

Embodiment 84. The method of embodiment 83, wherein the flowable cover medium is paraffin.

Embodiment 85. The method of embodiment 83 or 84, wherein the stained sample comprises a tissue stained with one or more of an immunohistochemical staining reagent, a labeled nucleic acid probe, or hematoxylin and/or eosin.

Embodiment 86. The method of any of embodiments 83 to 85, comprising dispensing molten paraffin as the flowable cover medium onto the stained sample.

Embodiment 87. The method of any of embodiments 83 to 86, comprising placing a paraffin infused sheet over the stained sample, wherein the flowable cover medium comprises the paraffin.

Embodiment 88. The method of any of embodiments 83 to 87, comprising immersing the stained sample in a bath comprising molten paraffin.

Embodiment 89. The method of any of embodiments 83 to 88, further comprising heating the covered sample to remove solvent from the sample while the flowable cover medium prevents exposure of the sample to oxygen.

Embodiment 90. The method of any of embodiments 83 to 89, further comprising adhering a coverslip to the covered sample.

Embodiment 91. The method of any of embodiments 83 to 90, further comprising storing the covered sample without a coverslip adhered to the slide.

Embodiment 92. The method of any of the foregoing embodiments, wherein the electric field is applied with a plurality of pins.

Embodiment 93. The method of any of the foregoing embodiments, wherein one or more pins are moved over a surface of the sample.

Embodiment 94. The method of any of the foregoing embodiments, further comprising:
removing paraffin from the sample without adding any liquid to the tissue.

Embodiment 95. The method of any of the foregoing embodiments, further comprising applying heat energy to the sample effective to melt the paraffin.

Embodiment 96. The method of embodiment 95, wherein the applying heat energy is performed by one or more methods selected from the group consisting of: placing the tissue sample on a surface and heating the surface; placing the tissue sample in a chamber and heating the chamber; applying radiant heat energy; applying infrared radiation; applying heat energy so as to heat the paraffin to a temperature in a range from about 35° C. to about 70° C.; and a combination of two or more of the foregoing.

Embodiment 97. The method of any of the foregoing embodiments, comprising applying the electric field so as to produce a plasma in an ionization region to which the sample is exposed.

Embodiment 98. The method of embodiment 97, wherein the plasma is produced from air molecules.

Embodiment 99. The method of any of the foregoing embodiments, wherein applying the electric field comprises applying a voltage between a first electrode and a second electrode.

Embodiment 100. The method of embodiment 99, wherein applying the electric field produces a corona discharge in a region surrounding the first electrode, and further comprising positioning the first electrode so as to expose the paraffin to the corona discharge.

Embodiment 101. The method of embodiment 99, wherein the first electrode has a configuration selected from the group consisting of:
the first electrode comprises a curved feature configured to generate a region of elevated electric field strength surrounding the first electrode; and
the first electrode comprises a curved feature configured to generate a region of elevated electric field strength surrounding the first electrode, wherein the curved feature comprises an edge or a tip of the first electrode.

Embodiment 102. The method of embodiment 99, wherein the second electrode is a planar electrode.

Embodiment 103. The method of embodiment 99, comprising moving at least one of the first electrode and the sample relative to the other.

Embodiment 104. The method of embodiment 99, wherein the first electrode comprises an array of electrodes.

Embodiment 105. The method of embodiment 99, wherein the tissue is positioned between the first electrode and the second electrode.

Embodiment 106. The method of embodiment 95, comprising applying the heat energy while applying the electric field.

Embodiment 107. The method of any of the foregoing embodiments comprising a step selected from the group consisting of: placing the sample on a solid substrate; placing the sample on a slide; placing the sample on a plate; placing the sample on beads; placing the sample on a porous medium; placing the sample on a filter; placing the sample in a liquid container; and placing the sample on a substrate surface having a higher surface energy than the paraffin.

It will be understood that terms such as “communicate” and “in communication with” (for example, a first component “communicates with” or “is in communication with” a second component) are used herein to indicate a structural, functional, mechanical, electrical, signal, optical, magnetic, electromagnetic, ionic or fluidic relationship between two or more components or elements. As such, the fact that one component is said to communicate with a second component is not intended to exclude the possibility that additional components may be present between, and/or operatively associated or engaged with, the first and second components.

It will be understood that various aspects or details of the invention may be changed without departing from the scope of the invention. Furthermore, the foregoing description is for the purpose of illustration only, and not for the purpose of limitation—the invention being defined by the claims.

1. A method of preparing a sample for in situ hybridization, the method comprising:
applying an electric field to a sample comprising a target polynucleotide; and
contacting the sample with one or more detectable nucleic acid probes which specifically bind to the target polynucleotide while the electric field is being applied.

2. The method of claim 1, wherein the sample is a paraffin-embedded tissue specimen.

3. The method of claim 1, wherein the sample is a whole tissue.

4. The method of claim 1, wherein the sample comprises a slice of whole tissue.

5. The method of claim 1, wherein the sample is one or more cell pellets.

6. The method of claim 1, wherein the sample is one or more cell smears.

7. The method of claim 1, wherein the sample comprises one or more cells and the target polynucleotide is within a nucleus of the cells.

8. The method of claim 1, wherein the electric field is applied at a strength and for a time sufficient to expose the DNA.

9. The method of claim 8, wherein the electric field is applied at a potential of 500V or less.

10. The method of claim 1, wherein the sample is not contacted with a proteolytic enzyme before the contacting with the nucleic acid probe.

11. The method of claim 10, wherein the sample is substantially free of autofluorescence from intact proteins during analysis of the labeled probes hybridized to the polynucleotide.

12. The method of claim 10, further comprising removing substantially all paraffin from the sample before the contacting with the labeled probes.

13. The method of claim 12, wherein the paraffin is removed by a combination of applying an electric field to the sample and rinsing the sample with a deparaffinization solvent.
